(19) United States
(12) Patent Application Publication
(10) Pub. No.: US 2026/0198437 A1
(43) Pub. Date: Jul. 16, 2026

(54) MULTIPLE-FLOWERING CUCURBITA PEPO SUBSP. PEPO PLANTS AND METHODS FOR THEIR PRODUCTION

(71) Applicant: The State of Israel, Ministry of Agriculture & Rural Development, Agricultural Research Organization, Rishon-LeZion (IL)

(72) Inventors: Amit GUR, Koranit (IL); Harry Stuart PARIS, Yokneam (IL); Galil TZURI, Kibbutz Gvat (IL)

(73) Assignee: The State of Israel, Ministry of Agriculture & Rural Development, Agricultural Research Organization, Rishon-LeZion (IL)

(21) Appl. No.: 19/632,577

(22) Filed: Mar. 30, 2026

Related U.S. Application Data
(63) Continuation of application No. PCT/IL2024/050964, filed on Sep. 27, 2024.
(60) Provisional application No. 63/540,981, filed on Sep. 28, 2023.

(51) Int. Cl. A01H 1/00; A01H 1/04; A01H 5/08; A01H 6/34

(52) U.S. Cl. CPC A01H 1/1215; A01H 1/045; A01H 5/08; A01H 6/348

(57) ABSTRACT

A Cucurbita pepo subsp. pepo plant is provided. The Cucurbita pepo subsp. pepo plant comprises a genome having a loss of function mutation in a Cp4.1LG13g07780 gene or ortholog thereof, wherein the loss of function mutation confers a multiflowering (mf) trait. Also provided are methods of producing and using such plants.

### RELATED APPLICATIONS

This application is a Continuation of PCT Patent Application No. PCT/IL2024/050964 having International filing date of Sep. 27, 2024 which claims the benefit of priority under 35 USC § 119(e) of U.S. Provisional Patent Application No. 63/540,981 filed on Sep. 28, 2023. The contents of the above applications are all incorporated by reference as if fully set forth herein in their entirety.

### SEQUENCE LISTING STATEMENT

The XML file, entitled 107292SequenceListing.xml, created on Mar. 26, 2026, comprising 71,637 bytes, submitted concurrently with the filing of this application is incorporated herein by reference.

### FIELD AND BACKGROUND OF THE INVENTION

The present invention, in some embodiments thereof, relates to Cucurbita pepo subsp. pepo plants and methods for their production.

Cucurbita pepo L. is one of the most widely grown species of vegetable crops. It is the most familiar species of Cucurbita L., a genus native to the Americas which encompasses plants bearing edible fruits known as pumpkins and squash. Like most other members of the Cucurbitaceae, Cucurbita plants are herbaceous, bearing large, palmate leaves and prominent fruits. Most species of Cucurbita are mesophytes, have fibrous root systems, and are monoecious, bearing large, intensely orange-yellow, nectar-producing, unisexual flowers that are foraged by bees. Each flower opens and is functional for one day and only during the early morning hours, withering by afternoon.

Cucurbita pepo is a collection of interfertile domesticated, feral, and wild plants. On the basis of allozyme variation and seed morphology, it has been classified into three subspecies, pepo, ovifera, and fraterna. Most cultivars belong to subsp. pepo but wild plants of this subspecies have not yet been found. The other cultivars belong to subsp. ovifera, which grows wild in central and southeastern U.S.A. Subsp. fraterna consists of wild specimens from northeastern Mexico, only.

Cucurbita pepo is perhaps the most polymorphic species in the plant kingdom. Its fruits range in size to over 20 kg; in shape from round to flat-scalloped, to long, bulbous cylindrical over 75 cm long; exterior color is based on hues of green, orange, and yellow, with color intensity ranging from pale to very intense, and gray contribution (darkness) ranging from none to very dark. Variegation, including striping and bicolor, can result in as many as four colors on the surface of the same fruit. Fruit mesocarp can be relatively thin or thick, and its color varies in the range from greenish white to white, yellow, light orange, and intense orange. Fruits rinds can be lignified or non-lignified, and smooth, warted, wrinkled, or netted.

Cucurbita pepo fruits are often used for culinary purposes when they are mature, 40 or more days past anthesis. However, the great economic value of this species rests on the common use of the young fruits, usually 2 to 5 days past anthesis, as food. These young fruits are known as summer squash. Summer squash are borne beginning approximately 50 days after seeding and as C. pepo grows well in a wide range of climates, it is very widespread in cultivation.

Each of the two cultivated subspecies contains four cultivar-groups (Groups) or “morphotypes” of edible-fruited cultivars, distinguished from one another on the basis of fruit shape. Most of the Groups are centuries old. Some, the Pumpkin, the Acorn, and the Scallop, are indeed quite old, having been bred by native Americans prior to the European contact at the end of the 15^{th} century. The Crookneck may also have been developed prior to the arrival of Europeans in North America. The Cocozelle and the Zucchini originated in southern and northern Italy, respectively. The Cocozelle is an old group, with records dating to the late 16^{th} century, and the Zucchini is the youngest group, with records dating only to the beginning of the late 19th century. The Cocozelle has some economic importance in Europe and in Israel yet, today, the Zucchini is by far the economically most important cultivar-group of Cucurbita pepo, perhaps exceeding in economic value the rest of the species, indeed, the rest of the genus combined.

Due to the high commercial value resting in the flowers and young fruits of Cucurbita pepo there is a need to develop new cultivars of this species having higher yields.

The multiple-flowering trait reported in Paris and Gur Euphytica volume 218, Article number: 19 (2022), is conferred by the single recessive gene, mf. This recessive gene was introgressed from a Crookneck squash (subsp. ovifera) into several inbreds of Cocozelle and Zucchini squash (subsp. pepo) through six backcross generations. The nearly isogenic hybrids derived from crossing these inbreds, single- and multiple-flowered, were compared with one another in replicated field trials with the purpose of determining whether or not the multiple-flowering trait can significantly increase yields of Cocozelle and Zucchini under field conditions. The results showed that the multiple-flowering trait can increase yield in Cocozelle squash by as much as 60% and in Zucchini squash by 24%. Identifying the location and the sequence of mf gene responsible for this trait would facilitate efficient generation of new varieties with high yields.

Additional background art includes:
WO2011/018785
Paris H S, Hanan A (2010) HortScience 45:1643-1644;
Paris and Gur Euphytica volume 218, Article number: 19 (2022);
Wang et al., (2015). A Rare SNP Identified a TCP Transcription Factor Essential for Tendril Development in Cucumber. Mol. Plant. 8, 1795-1808;
Luo et al., (1996). Origin of floral asymmetry in Antirrhinum. Nature 383, 794-799. doi: 10.1038/383 794a0;
Doebley et al., (1995). teosinte branched1 and the origin of maize: evidence for epistasis and the evolution of dominance. Genetics 141, 333-346;
Palatnik et al. (2003). Control of leaf morphogenesis by microRNAs. Nature 425, 257-263. doi: 10.1038/nature01958;
Braun et al. (2012). The Pea TCP transcription factor PsBRC1 acts downstream of strigolactones to control shoot branching. Plant Physiol. 158, 225-238. doi: 10.1104/pp. 111.182725;
Muhr et al., (2016). Knockdown of strigolactone biosynthesis genes in Populus affects BRANCHED1 expression and shoot architecture. New Phytol. 212, 613-626. doi: 10.1111/nph. 14076);
Nicolas et al., (2015). A recently evolved alternative splice site in the BRANCHED1a gene controls potato plant architecture. Curr. Biol. 25, 1799-1809. doi: 10.1016/j.cub.2015.05.053;
Mizuno et al. (2015). Chiba Tendril-Less locus determines tendril organ identity in melon (Cucumis melo L.) and potentially encodes a tendril-specific TCP homolog. J. Plant Res. 128, 941-951. doi: 10.1007/s10265-015-0747-2;
Ori, et al. Regulation of LANCEOLATE by miR319 is required for compound-leaf development in tomato. Nat Genet 39, 787-791 (2007).

### SUMMARY OF THE INVENTION

According to an aspect of some embodiments of the present invention there is provided a Cucurbita pepo subsp. pepo plant comprising a genome having a loss of function mutation in a Cp4.1LG13g07780 gene or ortholog thereof, wherein the loss of function mutation confers a multiflowering (mf) trait and wherein the plant is not of a variety, seeds of which, having been deposited at NCIMB Ltd. under Accession No. NCIMB 41744 (herein referred to as Multizuq) or progeny thereof or Accession No. NCIMB 41794 (herein referred to as Nizzan) or progeny thereof.

According to an aspect of some embodiments of the present invention there is provided a Cucurbita pepo subsp. pepo plant comprising a genome having a loss of function mutation in a Cp4.1LG13g07780 gene or ortholog thereof, wherein the loss of function mutation confers a multiflowering (mf) trait and wherein when the loss of function mutation is in an introgression from a Cucurbita pepo subsp. ovifera var. Crookneck ‘Supersett’, the introgression is smaller than 700 kb.

According to an aspect of some embodiments of the present invention there is provided a method of producing a Cucurbita pepo subsp. pepo plant having a multiflowering (mf) trait, the method comprising down-regulating a Cp4.1LG13g07780 gene or ortholog thereof in the Cucurbita pepo subsp. pepo plant to thereby produce a Cucurbita pepo subsp. pepo plant having the mf trait, and wherein when the down-regulating is by crossing with a Cucurbita pepo subsp. ovifera, an introgression resultant of the crossing is not that present in seeds having been deposited at NCIMB Ltd. under Accession No. NCIMB 41744 (herein referred to as Multizuq) or progeny thereof or Accession No. NCIMB 41794 (herein referred to as Nizzan) or progeny thereof.

According to an aspect of some embodiments of the present invention there is provided a method of producing a Cucurbita pepo subsp. pepo plant having a multiflowering (mf) trait, the method comprising down-regulating a Cp4.1LG13g07780 gene or ortholog thereof in the Cucurbita pepo subsp. pepo plant to thereby produce a Cucurbita pepo subsp. pepo plant having the mf trait, and wherein when the down-regulating is by crossing with a Cucurbita pepo subsp. ovifera, an introgression resultant of the crossing which comprises the ortholog of Cp4.1LG13g07780 is smaller than 700 kb.

According to an aspect of some embodiments of the present invention there is provided a method of identifying a Cucurbita pepo subsp. pepo plant useful for crossing, the method comprising identifying in a genome of the plant at least one nucleic acid sequence alteration indicative of a multiflowering (mf) trait located between MF1 and MF2 of chromosome 13 using marker assisted selection (MAS), wherein identification of the at least one nucleic acid sequence variation is indicative of Cucurbita pepo subsp. pepo plant useful for crossing.

According to an aspect of some embodiments of the present invention there is provided a method of identifying a Cucurbita pepo subsp. pepo plant useful for crossing, the method comprising identifying in a genome of the plant at least one nucleic acid sequence alteration indicative of a multiflowering (mf) trait located between MF21 and MF26 of chromosome 13 using marker assisted selection (MAS), wherein identification of the at least one nucleic acid sequence variation is indicative of Cucurbita pepo subsp. pepo plant useful for crossing.

According to an aspect of some embodiments of the present invention there is provided a method of processing a Cucurbita pepo subsp. pepo plant, the method comprising subjecting the plant as described herein to a process selected from the group consisting of cooking, baking, drying, extracting and frying thereby processing the Cucurbita pepo subsp. pepo plant.

According to an aspect of some embodiments of the present invention there is provided a processed product comprising DNA of the plant as described herein.

According to an aspect of some embodiments of the present invention there is provided a plant part comprising DNA of the plant as described herein.

According to some embodiments of the invention, the at least one loss of function mutation is in a homozygous form.

According to some embodiments of the invention, the down-regulating is by genome editing.

According to some embodiments of the invention, the down-regulating is by RNA silencing.

According to some embodiments of the invention, the down-regulating is by breeding with a plant of a Cucurbita pepo subsp. ovifera and backcrossing with the Cucurbita pepo subsp. pepo plant.

According to some embodiments of the invention, the down-regulating results in a loss of function mutation in the Cp4.1LG13g07780 gene or ortholog thereof.

According to some embodiments of the invention, the method further comprises validating presence of the loss-of-function mutation in the Cp4.1LG13g07780 gene or ortholog thereof.

According to some embodiments of the invention, the validating is effected using molecular markers.

According to some embodiments of the invention, the validating is effected biochemically by testing a function of the Cp4.1LG13g07780 gene or ortholog thereof.

According to some embodiments of the invention, the loss of function mutation or at least one nucleic acid sequence alteration is in Exon 1 of the Cp4.1LG13g07780 gene or ortholog thereof.

According to some embodiments of the invention, the loss of function mutation or at least one nucleic acid sequence alteration is in a YNNCNNFY sequence encoded by the gene or ortholog thereof.

According to some embodiments of the invention, the loss of function mutation or at least one nucleic acid sequence alteration is within Exon 1 of the gene or a corresponding position of an ortholog thereof.

According to some embodiments of the invention, the loss of function mutation or at least one nucleic acid sequence alteration at position 8,364,572 within Exon 1 of the gene or a corresponding position of an ortholog thereof is a frameshift mutation.

According to some embodiments of the invention, the Cucurbita pepo subsp. ovifera is of Group Crookneck.

According to some embodiments of the invention, the Cucurbita pepo subsp. pepo plant is of Group Zucchini or Cocozelle.

According to some embodiments of the invention, the loss of function mutation or at least one nucleic acid sequence alteration indicative of mf trait is located between MF21 and MF26 of chromosome 13.

According to some embodiments of the invention, the loss of function mutation or at least one nucleic acid sequence alteration indicative of mf trait is located between MF29 and MF26 of chromosome 13.

According to some embodiments of the invention, the loss of function mutation causes loss of tendrils identity regulation function.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D show the multi-flowering trait. (A) Illustration of the plant architecture of squash with a normal single flower per leaf axil (sf) and the recessive multiple-flowering phenotype (mf). Red arrows indicate the secondary bud in leaf axils of mf plant. (B) Images of the two female flowers in a single leaf axil of an mf Zucchini plant. (C) Effect of the multiple-flowering trait on total yield in nearly-isogenic Zucchini and Cocozelle squash varieties differing in the multiple flowering gene. (D) Effect of the multiple-flowering trait on number of marketable fruits in nearly-isogenic Zucchini and Cocozelle squash varieties differing in the multiple flowering gene.

FIGS. 2A-D show whole-genome mapping of MF trait—the strategy taken to map the multi-flowering (mf) trait. (A) The phenotypic segregation of the single-flowering (SF) and multiple-flowering (MF) phenotypes in two BC6F2 populations introgressed with the Crookneck MF allele into Cocozelle and Zucchini backgrounds. As shown in the pie-charts, the 1:3 MF:SF ratio is in line with the expected segregation of a single recessive gene. The Zucchini BC6F2 population (n=110) was used for mapping the mf trait using analysis of DNA bulks constructed from ~25 MF and ~25 SF segregants. (B) The results of a bulk-sequencing analysis (BSA-Seq) whereby a significant association is detected on chromosome 13. This whole-genome sequencing confirms the nearly-isogenic nature of the Zucchini BC6 line. (C) displays the SNP-index analysis at the chromosome 13 MF region. Based on this analysis, the trait interval is mapped to 7.80-8.80 Mbp based on the pattern of difference in allele frequencies between the MF and SF bulks. (D) The genomic profile of the narrow chr.13 ‘Superset’ introgression in the Cocozelle BC6 line characterized using specific PCR markers (MF07, MF17, MF13 and MF09, Table 4) developed based on parental polymorphisms.

FIGS. 3A-E shows positional cloning of the Cpmf gene. (A) Illustration of the F₂:3 recombinants between markers MF17 and MF9 used for the first round of substitution mapping to a ~28 Kb interval on Chromosome 13. (B) F_{2:3} recombinants between markers MF12 and MF19 used for the second round of substitution mapping to a ~6 Kb interval with a single gene, Cp4.1LG13g07780, annotated as TCP Transcription-factor DICHOTOMA-like. (C) F_{2:3} recombinants between markers MF29 and MF26 used for the third round of substitution mapping to a narrow 1,600 bp interval defining a single-base Insertion/Deletion (InDel) within the Cp4.1LG13g07780 gene (#10) as the most-probable causative sequence variant for the MF phenotype. The gene model is shown in its physical genomic coordinates. Red vertical numbered lines are non-synonymous polymorphisms between the SF and MF parental accessions. (D) Cp4.1LG13g07780 protein sequence and variants between MF (SET) and SF (TRF) parents. Polymorphism #10 is the single base-pair InDel causing frame-shift and altered protein sequence (in red) including within a conserved functional box (Yellow rectangle). The following SEQ ID Nos are indicated: SEQ ID NO: 10 is TCP protein of SET, SEQ ID NO: 11 is TCP protein of TRF, SEQ ID NO: 4 is TCP protein of Cp4.1LG13g07780 (reference termed also “07780 P”). The panel shows, variation in the protein sequence. Therefore, only the non-synonymous DNA polymorphisms affecting the predicted protein sequence are presented herein. Others are listed in Table 3 below. (E) shows a sequence alignment of the Cp4.1LG13g07780 TCP gene from the populations parents: Crookneck ‘Supersett’ (SET, MF donor), Zucchini ‘True French’ (TRF, SF) and Cocozelle Inbred 463 (SF). Shown are cDNA and gDNA alignments. Sequence variation between the accessions is indicated on the sequences. The following SEQ ID Nos are indicated: SEQ ID NO: 5 is TCP gDNA of TRF, SEQ ID NO: 6 is TCP gDNA of 463, SEQ ID NO: 7 is TCP gDNA of SET, SEQ ID NO: 8 is TCP cDNA of 463, SEQ ID NO: 9 is TCP cDNA of SET.

FIG. 4 shows a gel image of Cleaved Amplified Polymorphic Sequences (CAPS) marker developed for SNP #5 in the Cp4.1LG13g07780 gene (Table 4).

FIGS. 5A-C show that Cpmf gene is not differentially expressed between MF NILs, and is expressed specifically in tendrils and at the stem at leaf axil. (A) Comparison of expression of Cp4.1LG13g07780 (by qRT-PCR) between Zucchinni MF NILs across six tissues. (B) Comparison of expression of Cp4.1LG13g07780 (by qRT-PCR) between Cocozelle MF NILs across six tissues. (C) Comparison of expression of Cp4.1LG13g07780 (by RNA-Seq) between Zucchinni MF NILs across five tissues.

FIGS. 6A-B show that Cpmf is a TCP gene, homologous of the tendril (TEN) gene in melon and cucumber and share a common loss of tendrils identity function. (A) Modified leaf-like tendril in the Zucchinni (TRF) MF nearly-isogenic line. (B) Normal tendril in the Zucchinni (TRF) SF nearly-isogenic line.

### DESCRIPTION OF SPECIFIC EMBODIMENTS OF THE INVENTION

The present invention, in some embodiments thereof, relates to Cucurbita pepo subsp. pepo plants and methods for their production.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In C. pepo subsp. pepo one flower is produced per leaf axil whilst in C. pepo subsp. ovifera two or more flowers are produced per leaf axil. This multiple-flowering trait is conferred by a recessive gene designated mf, and the dominant allele, Mf, confers single-flowering (WO2011/018785 and Paris H S, Hanan A (2010) HortScience 45:1643-1644). The mf was introgressed from a Crookneck cultivar (subsp. ovifera) into a number of Cocozelle and Zucchini inbreds (subsp. pepo) (Paris 2017). It was found that the multiple-flowering trait can markedly increase yield of Cocozelle and Zucchini summer squash. Identification of the gene sequence and its location is of value to generate novel varieties with enhanced yield.

Thus, while reducing embodiments of the invention to practice, the present inventors have used two advanced backcross populations at the BC6F2 generation to identify the sequence and location of the gene responsible for the mf trait. MF is a single annotated gene within this interval, Cp4.1LG13g07780, annotated as TCP Transcription-factor DICHOTOMA-like. Several significant non-synonymous sequence polymorphisms were identified within this gene differentiating between the mapping population parents and between additional single- and multiple-flowering accessions. Most prominent is a single-base insertion causing frame-shift in translation and substantial change in the predicted sequence of this TCP protein (SNP #10).

The identification of mf as TCP gene associated in yield enhancement is surprising. The TCP gene family is characterized in other plants as responsible for different traits, such as branching or flower development, but not yield. In cucumber, for example, this gene is regulating tendril development, in Antirrhinum-flower morphology, side-branching in maize, leaf development in Arabidopsis, axillary buds outgrowth is peas and shoot architecture in poplar and potato and leaf morphology in tomato. Hence, this is the first annotation of this gene as responsible for the multi-flowering trait in Cucurbita pepo. Interestingly, this is the first report of a dual functionality for the TCP gene: in C. pepo the mutation in Cpmf not only induces meristematic activity to enable development of secondary buds but also modifies tendrils.

Thus, according to an aspect of the invention there is provided a Cucurbita pepo subsp. pepo plant comprising a genome having a loss of function mutation in a Cp4.1LG13g07780 gene or ortholog thereof, wherein the loss of function mutation confers a multiple-flowering (mf) trait and wherein when the loss of function mutation is in an introgression from a Cucurbita pepo subsp. ovifera var. crookneck ‘Supersett’, said introgression is smaller than 700 kb.

According to an aspect of the invention there is provided a Cucurbita pepo subsp. pepo plant comprising a genome having a loss of function mutation in a Cp4.1LG13g07780 gene or ortholog thereof, wherein the loss of function mutation confers a multiflowering (mf) trait and wherein the plant is not of a variety, seeds of which, having been deposited at NCIMB Ltd. under Accession No. NCIMB 41744 (herein referred to as Multizuq) or progeny thereof or Accession No. NCIMB 41794 (herein referred to as Nizzan) or progeny thereof. As used herein “Cucurbita pepo” refers to the collection of interfertile domesticated, feral and wild plants of the subspecies pepo, ovifera, and fraterna.

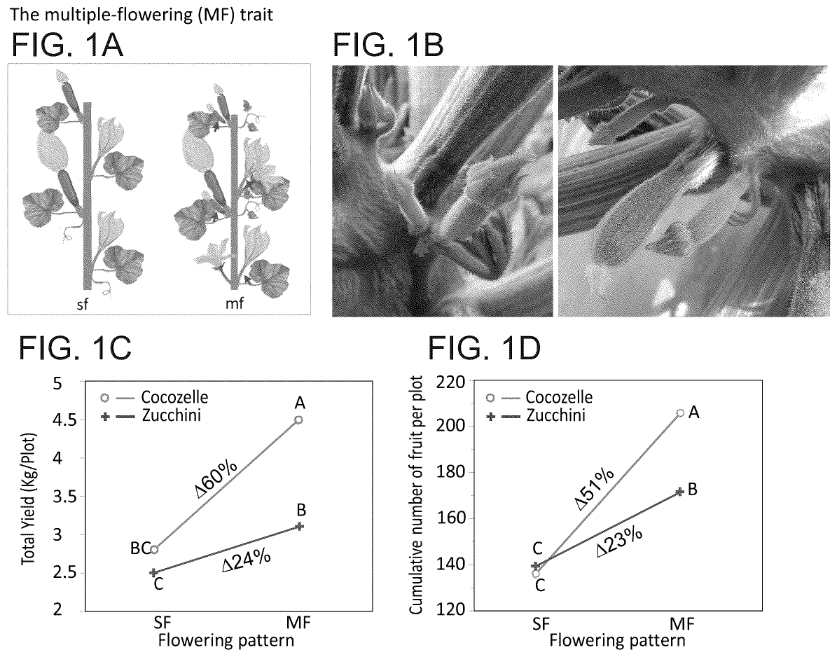

On the basis of fruit shape, Cucurbita pepo is considered to comprise eight edible-fruited cultivar-groups, also known as morphotypes (FIGS. 1, 2, and 3 of WO2011/018785), four of which are classified in C. pepo subsp. pepo and the other four in C. pepo subsp. ovifera. Description of the eight Groups and their placement in the two cultivated subspecies are summarized in Table 1, below. The fruits of two of the cultivar-groups, Pumpkin and Acorn, are used primarily when mature. Pumpkin fruits are round, being spherical, oblate, globular, or oval and Acorn fruits are turbinate with alternating longitudinal ridges and furrows. In both of these groups, the length-to-width ratio of the fruits is approximately 1:1. The fruits of the other six cultivar-groups are used when immature, as summer squash, and diverge markedly from this 1:1 ratio. Scallop squash are flat and scalloped, hence having a length-to-width ratio that is considerably less than 1:1. The other five groups, Cocozelle, Crookneck, Straightneck, Vegetable Marrow, and Zucchini, have a length-to-width ratio that is considerably greater than 1:1 (Table 1, below). Each cultivar-group of both subspecies is comprised of numerous cultivars. The name of one representative cultivar of each cultivar-group is given in Table 1.

TABLE 1
The edible-fruited cultivar-groups of Cucurbita pepo (after Paris, H.S. (2000) History of
the cultivar-groups of Cucurbita pepo. Horticultural Reviews 25: 71-170).

| Cultivar- | | | Standard | |
|---|---|---|---|---|
| group | Synonyms | Subspecies | cultivar | Fruit shape |
| Pumpkin | | pepo | Connecticut | Round: spherical oblate, ovate, globular |
| | | | Field | |
| Vegetable | Middle | pepo | Beirut | Short, tapered cylindrical, narrow at |
| Marrow | eastern | | | peduncle end, broad at stylar end, |
| | | | | length-to-broadest width ratio ranging |
| | | | | from 1.5-3.0 |
| Cocozelle | Italian | pepo | Striato d'Italia | Long to extremely long, cylindrical, |
| | | | | bulbous near stylar end, length-to-broadest |
| | | | | width ratio at least 3.5 |
| Zucchini | Courgette | pepo | Black Zucchini | Uniformly cylindrical, length-to-broadest |
| | | | | width ratio 3.5-5.0 |
| Acorn | Table | ovifera | Table Queen | Turbinate, ridged and furrowed, broad at |
| | Queen | | | peduncle end, convex at stylar end |
| Crookneck | | ovifera | Yellow | Elongated, with narrow, slightly to very |
| | | | Summer | curved neck, broad stylar half, convex |
| | | | Crookneck | stylar end |
| Scallop | Patty Pan, | ovifera | Golden Bush | Flattened, with scalloped margins |
| | Patisson | | Scallop | |
| Straightneck | | ovifera | Early Prolific | Cylindrical, with short neck or |
| | | | Straightneck | constriction near the stem end and broad |
| | | | | stylar half, convex or pointed distal end |

The term “‘plant” as used herein encompasses whole plants and plant parts, including seeds, cuttings, shoots, stems, roots, flowers, buds, and plant cells, tissues and organs. The plant may be in any form including suspension cultures, embryos, meristematic regions, callus tissue, leaves, gametophytes, sporophytes, pollen, and microspores.

Also included are, ancestors and progeny of the plants.

According to a specific embodiment, the plant is a progeny which comprises the loss of function mutation as described herein.

As used herein, “Cucurbita pepo subsp. pepo” is a subspecies of the species Cucurbita pepo, which is commonly known as the pumpkin or summer squash. This subspecies is widely cultivated and consumed around the world. It includes four edible-fruited cultivar-groups (morphotypes):
Pumpkin: Fruits round, from spherical, globular, oblate, or oval. Cultivars include Connecticut Field, Small Sugar, Jack O'Lantern, Spookie, Howden's Field, Lady Godiva;
Vegetable Marrow: Short, tapered cylindrical, narrow at peduncle end, broad at stylar end, length-to-broadest width ratio ranging from 1.5-3.0. Cultivars include Beirut, Vegetable Marrow, Vegetable Spaghetti;
Cocozelle: Long to extremely long, cylindrical, bulbous near stylar end, length-to-broadest width ratio at least 3.5. Cultivars include Striato d'Italia, Cocozelle, San Pasquale, Romanesco, Striato Pugliese, Lungo Fiorentino;
Zucchini: Uniformly cylindrical, length-to-broadest width ratio 3.5-5.0. Cultivars include Black Zucchini, Fordhook Zucchini, True French, Zucchini, Nano Verde di Milano.

As used herein, “Cucurbita pepo subsp. ovifera” is a subspecies of the species Cucurbita pepo, which is commonly known as winter squash or summer squash. This subspecies is widely cultivated and consumed in North America, less so elsewhere. It includes four edible-fruited cultivar-groups (morphotypes):
Scallop or Patty Pan or Patisson: Flattened, with scalloped margins. Cultivars include Golden Bush Scallop, White Bush Scallop, Yellow Bush Scallop, Benning's Green Tint, Peter Pan;
Acorn: Turbinate, ridged and furrowed, broad at peduncle end, convex at stylar end. Cultivars include Table Queen, Table King, Table Gold, Carnival, Thelma Sanders;
Crookneck: Elongated, with narrow, slightly to very curved neck, broad stylar half, convex stylar end. Cultivars include Yellow Summer Crookneck, Early Golden Crookneck, Dixie, Supersett;
Straightneck: Cylindrical, with short neck or constriction near the stem end and broad stylar half, convex or pointed distal end. Cultivars include Early Prolific Straightneck, Straightneck Early, Seneca Butterbar.

According to a specific embodiment, the Cucurbita pepo subsp. pepo is Zucchini, e.g., True French.

According to a specific embodiment, the Cucurbita pepo subsp. pepo is Cocozelle, e.g., Striato d'Italia.

As used herein “C. pepo subsp. ovifera” is a subspecies of the species Cucurbita pepo. This subspecies is native to North America, particularly in the southern and central United States, including Texas, Arkansas, Missouri, Illinois, and Mississippi. Wild members of this subspecies produce small, round or pear-shaped fruits. These fruits are typically not cultivated for consumption like other Cucurbita pepo varieties such as zucchinis, cocozelles, or pumpkins. Instead, they are often grown for their ornamental value or used in traditional Native American crafts. The gourds have a hard, durable shell and are used to create decorative items, such as rattles, containers, and ornaments. Cultivated members of this subspecies have larger fruits of various shapes.

According to a specific embodiment, the Cucurbita pepo subsp. ovifera is var. crookneck ‘Supersett’.

‘Multizuq’, is the F1 hybrid of zucchini breeding line 1688-1-3-16 crossed with zucchini breeding line 1477-1-7-2-10. Seeds of ‘Multizuq’ were deposited under the Budapest treaty on Jul. 30, 2010 at the NCIMB Ltd. Scotland UK, under the accession number NCIMB 41744.

‘Nizzan’, is the F1 hybrid of cocozelle breeding line 1260-4-6-2-10 crossed with cocozelle breeding line 1413-4-54-7. Seeds of Nizzan were deposited under the Budapest treaty on Dec. 10, 2010 at the NCIMB Ltd. Scotland UK, under the accession number 41794.

As used herein “progeny” refers to breeding products of a given plant (e.g., variety), offspring or descendants thereof.

Plants of the invention have more than one flower/fruit per node. Flower buds in Cucurbita pepo are differentiated in or beside the leaf axils, that is, at the junctions of the petiole bases with the stem; these junctions are also referred to as stem nodes. Thus, according to embodiments of the invention, 2, 3, 4 or even more flower buds can be formed at each stem node.

As mentioned, plants of the invention produce more than one flower at most stem nodes and have fruits which are endowed with the phenotype of Cucurbita pepo subsp. pepo. The potentially higher yield of flowers and fruits per hybrid plant as compared to all previously existing Cucurbita pepo subsp. pepo does not compromise fruit phenotype.

As used herein the term “multiple-flowering”, “multi-flowering”, “multi-flowering trait”, “mf” or “the trait” refers to the production of two or more flowers or fruit per leaf axil (also referred to herein as “node”).

The advantage of the multi-flowering trait is more flowers (at least 2 per axil) and increased fruit yields. The multi-flowering trait is expressed at most leaf axils (i.e., more than 50% of the leaf axils), the fruit of the plant having the phenotype of that of Cucurbita pepo subsp. pepo.

As used herein “increased yield” refers to a statistically significant increase of fruit yield or flowers of more than 10%, 20%, 30%, 40%, 50%, 60%, 70%, 90%, 2 fold, 3 fold or more than compared to that of the same Cucurbita pepo subsp. pepo which does not have the loss of function mutation in TCP.

According to a specific embodiment, the yield is measured by fruit weight per growth area, e.g., kg/plot.

According to a specific embodiment, the yield is measured as cumulative number of fruit per plant (per season).

According to a specific embodiment, the yield is measured as cumulative number of flowers per plant (per season).

According to a specific embodiment, the flowers refers to male flowers.

According to a specific embodiment, the flowers refers to female flowers.

According to a specific embodiment, the flowers refers to male flowers and female numbers.

Of note, the mf gene is synonymous to the Cp4.1LG13g07780 TCP gene.

As mentioned, the Cucurbita pepo subsp. pepo plant comprises a genome, which means that more than 99% of the genome is that of Cucurbita pepo subsp. pepo but in some cases not a 100% of the genome is that of Cucurbita pepo subsp. pepo. This can happen when foreign genomic segments are introduced into the genome such as by crossing or transgenesis.

As used herein “a Cp4.1LG13g07780 gene” is the DNA, RNA or protein product of the Cp4.1LG13g07780 gene (SEQ ID NOs: 1-4). See CuGenDB Cp4.1LG13g07780 (gene) Cucurbita pepo (MU-CU-16) v4.1 http://cucurbitgenomics(dot)org/v2/feature/gene/Cp4.1LG13g07780.

Genomic positions of Cp4.1LG13g07780: Chromosome 13: Exon1: U.S. Pat. Nos. 8,363,831-8,364,788: Intro1: U.S. Pat. Nos. 8,364,789-8,364,883; Exon2: U.S. Pat. Nos. 8,364,884-8,364,945.

As used herein “ortholog” refers to the homolog (alleles) of the gene found (above 80%, 90%, 95% identity to Cp4.1LG13g07780) in a different Cucurbita pepo subsp. pepo or texana (the latter being with a loss of function mutation species but related by linear descent) and affecting the mf trait. The ortholog typically shares the same genomic structure as that of Cp4.1LG13g07780. A person skilled in the art will know what the genomic structure of the ortholog including exon and intron localization.

Exemplary orthologs for mf gene Cucurbita pepo subsp. pepo or ovifera are shown in Table 2. The present teachings, in some embodiments thereof, refer to the manipulation of the Cucurbita pepo subsp. pepo orthologs, as in Table 2 below.

TABLE 2

| | | | | | | | EXON 1 |
|---|---|---|---|---|---|---|---|
| | | | | | | | 8,364,142 |
| | | | | Morpho | | | SNP#5 |
| | | | | type - | | | [G/A] |
| Sample | | | Short | Cultivar | Accession | Flowering | (CAPS |
| # | Name | ssp. | Group | Group | Name | Pattern | Marker) |
| A1 | SET | Ovifera | CN | Crookneck | O-CN-SET | MF | G |
| A2 | TRUE FRENCH | Pepo | ZU | Zucchini | P-ZU-TRF | SF | A |
| A3 | TABLE KING | Ovifera | AC | Acorn | O-AC-TKI | MF | G |
| A4 | SWEET | Ovifera | AC | Acorn | O-AC-SWD | MF | G |
| | DUMPLING | | | | | | |
| A5 | BELYE 13 | Ovifera | SC | Scallop | O-SC-BEY | MF | G |
| A6 | GOLDEN | Ovifera | SC | Scallop | O-SC-GBS | MF | G |
| | BUSH SCALOP | | | | | | |
| A8 | JACK | Pepo | PU | Pumpkin | P-PU-JOL | SF | A |
| | O'LANTERN | | | | | | |
| A9 | UKRAINSKA | Pepo | PU | Pumpkin | P-PU-UNO | SF | A |
| | NOGOPLODNA | | | | | | |
| A10 | ROMANESCO | Pepo | CO | Cocozelle | P-CO-ROM | SF | A |
| A11 | PRECOCIOUS | Pepo | ZU | Zucchini | P-ZU-PFZ | SF | A |
| | FORDHOOK | | | | | | |
| | ZUCCHINI | | | | | | |
| A12 | INDIA | Pepo | CO | Cocozelle | P-CO-INN | SF | A |
| A13 | PI491201 | Pepo | VM | veg. Marrow | P-VM- | SF | A |
| | | | | | PI491201 | | |
| A14 | IPK1575 | Pepo | PU | Pumpkin | P-PU-1575 | SF | A |
| A15 | 85a | Pepo | VM | veg. Marrow | P-VM-85a | SF | A |
| A16 | PETER PAN | Ovifera | SC | Scallop | O-SC-PPN | MF | G |
| | INBRED | | | | | | |
| B1 | Table Queen | Ovifera | AC | Acorn | TQE | MF | G |
| B2 | Mammoth Table | Ovifera | AC | Acorn | MTQ | MF | G |
| | Queen | | | | | | |
| B3 | Jersey Golden | Ovifera | AC | Acorn | JGA | MF | G |
| | Acorn | | | | | | |
| B4 | Early Crookneck | Ovifera | CN | Crookneck | EAC | MF | G |
| B5 | Yellow Summer | Ovifera | CN | Crookneck | YSC | MF | G |
| | Crookneck | | | | | | |
| B6 | Benning's Green | Ovifera | SC | Scallop | BGT | MF | G |
| | Tint | | | | | | |
| B7 | White Bush | Ovifera | SC | Scallop | WBS | MF | G |
| | Scallop | | | | | | |
| B8 | Yellow Bush | Ovifera | SC | Scallop | YBS | MF | G |
| | Scallop | | | | | | |
| B9 | Early Prolific | Ovifera | SN | Straightneck | EPS | MF | G |
| | Straightneck | | | | | | |
| B11 | Lungo Bianco di | Pepo | CO | Cocozelle | LBS | SF | A |
| | Sicilia | | | | | | |
| B12 | San Pasquale | Pepo | CO | Cocozelle | SPQ | SF | A |
| B15 | PI 311103 | Pepo | PU | Pumpkin | PI 311103 | SF | A |
| | Guicoy | | | | | | |
| B17 | Beirut | Pepo | VM | veg. Marrow | BEI | SF | A |
| B18 | Vegetable | Pepo | VM | veg. Marrow | VGM | SF | A |
| | Marrow | | | | | | |
| B19 | Black Zucchini | Pepo | ZU | Zucchini | BZU | SF | A |
| B20 | Nero di Milano | Pepo | ZU | Zucchini | NER | SF | A |
| B21 | Striped Pear | Ovifera | GO | | SPR | MF | G |
| | (Bicolor Pear) | | | | | | |

As used herein, the phrase “loss-of-function mutation” refers to at least one mutation in the DNA sequence of a gene (in this case a Cp4.1LG13g07780 gene or ortholog thereof), which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity or which sentence the protein to degradation; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an Indel—a mutation which can be either an insertion or a deletion of bases in the genome of an organism. Indels ≥50 bases in length or an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift. The mutation can be in a coding region or a non-coding region.

According to specific embodiments, the loss of function mutation is selected from the group consisting of an insertion, a deletion, an insertion/deletion (indel) and a substitution.

According to specific embodiments, the loss of function mutation is a frameshift mutation.

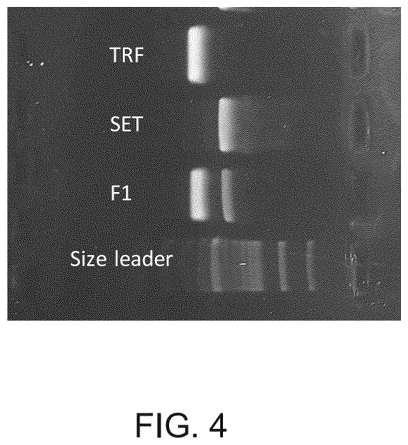

According to a specific embodiment, the loss-of-function mutation affects the translated protein sequence. See for example FIG. 4.

TABLE 3

| | SNP # | | | 1 | 2 | 3 | 4 | 5 | 6 | 7 | 8 | 9 | 10 |
|---|---|---|---|---|---|---|---|---|---|---|---|---|---|
| | Exon/Intron | | | EXON | EXON | EXON | EXON | EXON | EXON | EXON | EXON | EXON | EXON |
| | | | | 1 | 1 | 1 | 1 | 1 | 1 | 1 | 1 | 1 | 1 |
| | Genomic | | | 8,363,856 | 8,363,869 | 8,364,032 | 8,364,098 | 8,364,142 | 8,364,337 | 8,364,433 | 8,364,497 | 8,364,514 | 8,364,572 |
| | position - | | | | | | | | | | | | |
| | Chr 13 | | | | | | | | | | | | |
| | Polymorphism | | | SNP | SNP | insertion | SNP | SNP | SNP | SNP | Insertion | SNP | insertion |
| | type | | | [A/G] | [T/A] | (AAG) | [C/T] | [A/G] | [C/T] | [T/A] | (CCGCCA) | [T/A] | (C) |
| Sample# | Predicted | | | (AA) | no | additional | (AA) | no | no | no | additional | no | frame |
| | Effect on | | | His- | | 1-4 Lys | Ser - | | | | 2/4 | | shift |
| | protein | | | Arg | | | Pro | | | | Proline | | |
| | Accession | Sub- | Flowering | | | | | | | | | | |
| | | species | pattern | | | | | | | | | | |
| A1 | CN SET | Ovifera | MF | G | A | (AAG) | T | G | C | A | (CCGCCA)1 | A | ✓ |
| | | | | | | 3 | | | | | | | |
| A2 | ZU TRF | Pepo | SF | A | T | | C | A | C | T | | T | |
| A3 | O-AC-TKI | Ovifera | MF | G | A | (AAG) | T | G | C | T | (CCGCCA)2 | A | |
| | | | | | | 1 | | | | | | | |
| A4 | O-AC-SWD | Ovifera | MF | G | A | (AAG) | T | G | C | T | (CCGCCA)2 | A | |
| | | | | | | 2 | | | | | | | |
| A5 | O-SC-BEY | Ovifera | MF | G | A | (AAG) | T | G | C | A | (CCGCCA)1 | A | ✓ |
| | | | | | | 3 | | | | | | | |
| A6 | O-SC-GBS | Ovifera | MF | G | A | (AAG) | T | G | C | A | (CCGCCA)1 | A | ✓ |
| | | | | | | 4 | | | | | | | |
| A8 | P-PU-JOL | Pepo | SF | A | T | | C | A | C | T | | T | |
| A9 | P-PU-UNO | Pepo | SF | A | T | | C | A | C | T | | T | |
| A10 | P-CO-ROM | Pepo | SF | A | T | | C | A | C | T | | T | |
| A11 | P-ZU-PFZ | Pepo | SF | A | T | | C | A | C | T | | T | |
| A12 | P-CO-INN | Pepo | SF | A | T | | C | A | C | T | | T | |
| A13 | P-VM- | Pepo | SF | A | T | | C | A | C | T | | T | |
| | PI491201 | | | | | | | | | | | | |
| A14 | P-PU- | Pepo | SF | A | T | | C | A | C | T | | T | |
| | IPK1575 | | | | | | | | | | | | |
| A15 | P-VM-85a | Pepo | SF | A | T | | C | A | T | T | | T | |
| A16 | O-SC-PAN | Ovifera | MF | G | A | (AAG) | T | G | C | A | (CCGCCA)1 | A | ✓ |
| | | | | | | 4 | | | | | | | |

| | SNP # | | | 11 | 12 | 13 | 14 | 15 | 16 | 17 | 18 |
|---|---|---|---|---|---|---|---|---|---|---|---|
| | Exon/Intron | | | EXON 1 | INTRON | INTRON | INTRON | INTRON | INTRON | INTRON | INTRON |
| | Genomic | | | 8,364,681 | 8,364,790 | 8,364,796 | 8,364,799 | 8,364,805 | 8364780-815 | 8,364,813 | 8,364,831 |
| | position - | | | | | | | | | | |
| | Chr 13 | | | | | | | | | | |
| | Polymorphism | | | SNP | Deletion | SNP | SNP | SNP | Deletion | INDEL | SNP |
| | type | | | [A/C] | (TT) | [A/T] | [T/A] | [T/A] | (15 bp) | (A/TT) | [G/A] |
| Sample# | Predicted | (AA) ASN-THR | | | NO | NO | NO | NO | NO | NO | NO |
| | Effect on | | | | | | | | | | |
| | protein | | | | | | | | | | |
| | Accession | Sub- | Flowering | | | | | | | | |
| | | species | pattern | | | | | | | | |
| A1 | CN SET | Ovifera | MF | A | ✓ | A | A | A | | TT | G |
| A2 | ZU TRF | Pepo | SF | A | | A | T | T | | A | G |
| A3 | O-AC-TKI | Ovifera | MF | C | ✓ | T | — | — | ✓ | TT | A |
| A4 | O-AC-SWD | Ovifera | MF | C | ✓ | T | — | — | ✓ | TT | A |
| A5 | O-SC-BEY | Ovifera | MF | A | ✓ | A | A | A | | TT | G |
| A6 | O-SC-GBS | Ovifera | MF | A | ✓ | A | A | A | | TT | G |
| A8 | P-PU-JOL | Pepo | SF | A | | A | T | T | | A | G |
| A9 | P-PU-UNO | Pepo | SF | A | | A | T | T | | A | G |
| A10 | P-CO-ROM | Pepo | SF | A | | A | T | T | | A | G |
| A11 | P-ZU-PFZ | Pepo | SF | A | | A | T | T | | A | G |
| A12 | P-CO-INN | Pepo | SF | A | | A | T | T | | A | G |
| A13 | P-VM- | Pepo | SF | A | | A | T | T | | A | G |
| | PI491201 | | | | | | | | | | |
| A14 | P-PU- | Pepo | SF | A | | A | T | T | | A | G |
| | IPK1575 | | | | | | | | | | |
| A15 | P-VM-85a | Pepo | SF | A | | A | T | T | | A | G |
| A16 | O-SC-PAN | Ovifera | MF | A | ✓ | A | A | A | | TT | G |

According to specific embodiments, the loss-of-function mutation of the gene may comprise at least one allele of the gene.

It will be appreciated that since the gene is a single gene dominating the trait, in order to have an adequate effect, both alleles should comprise a loss of function mutation (not necessarily the same in a homozygous form), but a single allele mutation can be also useful for breeding and research hence both are contemplated herein.

According to specific embodiments, the loss of function mutation is in both alleles of the genome.

According to specific embodiments, the loss of function mutation is in a homozygous form.

The term “allele” as used herein, refers to any of one or more alternative forms of a gene locus, which alleles relate to a trait or characteristic. In a diploid Cucurbita pepo, the two alleles of a given gene occupy corresponding loci on a pair of homologous chromosomes.

According to other specific embodiments a loss-of-function mutation of a gene comprises both alleles of the gene. In such instances the e.g. Cp4.1LG13g07780 may be in a homozygous form or in a heterozygous form. According to this embodiment, homozygosity is a condition where both alleles of the gene e.g. Cp4.1LG13g07780 are characterized by the same nucleotide sequence. Heterozygosity refers to different conditions of the gene at the e.g. Cp4.1LG13g07780 gene.

According to a specific embodiment the loss of function mutation is in a homozygous or heterozygous form yet both encode for dis-functioning products.

According to a specific embodiment, the loss of function mutation refers to at least one Indel.

According to a specific embodiment, the loss of function mutation causes a premature stop codon.

According to a specific embodiment, the loss of function mutation causes a frameshift.

According to a specific embodiment, the loss of function mutation is in Exon 1 of said Cp4.1LG13g07780 gene or ortholog thereof.

According to a specific embodiment, the loss of function mutation is in a YNNCNNFY (SEQ ID NO: 13) sequence of the gene or ortholog thereof.

According to a specific embodiment, the loss of function mutation encompasses the position of SNP #10, however the identity of the mutation can be different (e.g., as detailed above).

According to a specific embodiment, the mutation retains the protein product but abolishes its activity e.g., transcription factor activity.

According to a specific embodiment, the mutation affects the level or protein expression, e.g., to such that is undetectable at the mRNA and/or protein level as can be determined by RT-PCR or Western blot.

According to a specific embodiment, the genetic modification reduces the level of expression and/or activity of the gene by at least 80% (e.g., 81%, 82%, 83%, 84%, 85%, 86%, 87%, 88%, 89%, 90%, 91%, 92%, 93%, 94%, 95%, 96%, 97%, 98%, 99% or 100%).

According to a specific embodiment, the genetic modification reduces the level of expression of the gene by at least 80% (e.g., 81%, 82%, 83%, 84%, 85%, 86%, 87%, 88%, 89%, 90%, 91%, 92%, 93%, 94%, 95%, 96%, 97%, 98%, 99% or 100%).

The level of expression can be affected by the level of transcription, translation or the formation of aberrant protein products which are typically sentenced to degradation.

The present invention also envisages in some embodiments thereof, a Cucurbita pepo subsp. pepo plant having been treated with an agent (e.g., RNA silencing agent) to reduce the level of expression or activity of Cp4.1LG13g07780 gene or ortholog thereof, as compared to that of a Cucurbita pepo subsp. pepo plant of the same age and genetic background not having been treated with such an agent. Details on how to obtain such a plant are provided hereinbelow.

The comparison can be made to an identical tissue of a Cucurbita pepo subsp. pepo plant of the same genetic background and developmental stage,

The “same genetic background” refers to at least 99% or 99.9% of the genome is shared between the plant and the non-mutated plant (both of Cucurbita pepo subsp. pepo).

According to a specific embodiment, the plant is non-transgenic.

According to a specific embodiment, the plant may be a transgenic plant. For example, the transgene may function to improve biotic stress resistance, pesticide resistance or abiotic stress resistance. In an alternative example, the plant may be transgenic to a genome editing agent, however such is usually discarded later on, such as by further breeding or self-editing mechanisms that remove the genome editing agents (such as Cas9). Examples are provided in WO2021/100034 which is hereby incorporated by reference in its entirety.

According to a specific embodiment, the plant is a hybrid plant or the seed is a hybrid seed, where e.g., each of the parental lines is homozygous for a loss-of-function mutation in the gene as described herein.

As used herein the term “hybrid” refers to the offspring derived from crossing two parental breeding lines of Cucurbita pepo.

The hybrid is heterozygous and derived from the crossing of the two parental breeding lines, each of which is nearly homozygous. The hybrid and each of its two parent breeding lines are homogeneous populations. The hybrid and its parents according to the invention contain, however, at least a small portion of the genome, by introgression, of Cucurbita pepo subsp. ovifera, specifically, the gene or genes conferring the production of more than one flower/fruit per node and adjacent chromosomal regions.

Methods of producing the plant as described herein may rely on the use of mutagens e.g., EMS or breeding or genetic engineering, e.g., genome editing, which is naturally a more directed method and therefore negates the need for breeding steps.

According to an aspect of the invention there is provided a method of producing a Cucurbita pepo subsp. pepo plant having a multiflowering (mf) trait, the method comprising down-regulating a Cp4.1LG13g07780 gene or ortholog thereof in the Cucurbita pepo subsp. pepo plant to thereby produce a Cucurbita pepo subsp. pepo plant having the mf trait, and wherein when said down-regulating is by crossing with a Cucurbita pepo subsp. ovifera, an introgression resultant of said crossing is not that present in seeds having been deposited at NCIMB Ltd. under Accession No. NCIMB 41744 (herein referred to as Multizuq) or progeny thereof or Accession No. NCIMB 41794 (herein referred to as Nizzan) or progeny thereof.

According to an aspect of the invention there is provided a method of producing a Cucurbita pepo subsp. pepo plant having a multi-flowering (mf) trait, the method comprising down-regulating a Cp4.1LG13g07780 gene or ortholog thereof in the Cucurbita pepo subsp. pepo plant to thereby produce a Cucurbita pepo subsp. pepo plant having the mf trait, and wherein when said down-regulating is by crossing with a Cucurbita pepo subsp. ovifera (e.g., Crookneck Group, ‘Supersett’), an introgression resultant of said crossing which comprises said ortholog of Cp4.1LG13g07780 is smaller than 700 kb (e.g., smaller than 50 kb, 300 kb, 200 kb, 100 kb).

According to a specific embodiment, the genetically modifying is by breeding with a Cucurbita pepo subsp. ovifera.

According to a specific embodiment, the genetically modifying is not by breeding.

According to a specific embodiment, the genetically modifying is by breeding but not with a Cucurbita pepo subsp. ovifera, e.g., Crookneck Group e.g., Supersett.

According to another specific embodiment, the genetically modifying is by genome editing.

According to another specific embodiment, the genetically modifying is in both alleles of the gene or ortholog thereof.

Below is a description of platform technologies for effecting knock-out (also referred to as “genome editing”) and transcriptional silencing in plants.

Methods of introducing nucleic acid alterations to a gene of interest are well known in the art [see for example Andolflo et al. Advances in Plant Breeding Strategies: Vegetable Crops pp 407-422; Ahmar el al. ront. Plant Sci., 28 May 2021 Sec. Plant Biotechnology volume 12-2021 1www(dot)doi(dot)org/10.3389/fpls.2021.663849)].

Following is a description of various exemplary methods used to introduce nucleic acid alterations to a gene of interest and agents for implementing same that can be used according to specific embodiments of the present invention.

Any of the below methods can be directed to any part of the gene (e.g., Cp4.1LG13g07780) as long as a loss-of-function is achieved.

As used herein “target sequence” refers to the Cp4.1LG13g07780 gene or ortholog DNA or RNA transcript.

Contemplated herein, according to some embodiments, any CRISPR-Cas modification that will introduce early stop-codon or a frame shift as in InDel #10. Modification of the conserved amino-acids sequences starting at the end of exon 1 and extend all the way to the C terminus of the predicted protein, as shown in FIG. 4 (references for conserved boxes Wang et al. 2015 2015. A Rare SNP Identified a TCP Transcription Factor Essential for Tendril Development in Cucumber. Molecular Plant 8, 1795-1808, Zhang et al 2022 Polyploidy events shaped the expansion of transcription factors in Cucurbitaceae and exploitation of genes for tendril development. Horticultural Plant Journal 8, 562-574).

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

Another agent capable of downregulating Cp4.1LG13g07780 gene or ortholog is a RNA-guided endonuclease technology e.g. CRISPR system (that is exemplified in great details in the Examples section which follows).

As used herein, the term “CRISPR system” also known as Clustered Regularly Interspaced Short Palindromic Repeats refers collectively to transcripts and other elements involved in the expression of or directing the activity of CRISPR-associated genes, including sequences encoding a Cas9 gene (e.g. CRISPR-associated endonuclease 9), a tracr (trans-activating CRISPR) sequence (e.g. tracrRNA or an active partial tracrRNA), a tracr-mate sequence (encompassing a “direct repeat” and a tracrRNA-processed partial direct repeat) or a guide sequence (also referred to as a “spacer”) including but not limited to a crRNA sequence (i.e. an endogenous bacterial RNA that confers target specificity yet requires tracrRNA to bind to Cas) or a sgRNA sequence (i.e. single guide RNA).

In some embodiments, one or more elements of a CRISPR system is derived from a type I, type II, or type III CRISPR system. In some embodiments, one or more elements of a CRISPR system (e.g. Cas) is derived from a particular organism comprising an endogenous CRISPR system, such as Streptococcus pyogenes, Neisseria meningitides, Streptococcus thermophilus or Treponema denticola.

In general, a CRISPR system is characterized by elements that promote the formation of a CRISPR complex at the site of a target sequence (also referred to as a protospacer in the context of an endogenous CRISPR system).

In the context of formation of a CRISPR complex, “target sequence” (in this case a Cp4.1LG13g07780 gene or ortholog thereof) refers to a sequence to which a guide sequence (i.e. guide RNA e.g. sgRNA or crRNA) is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. Thus, according to some embodiments, global homology to the target sequence may be of 50%, 60%, 70%, 75%, 80%, 85%, 90%, 95% or 99%. A target sequence may comprise any polynucleotide, such as DNA or RNA polynucleotides. In some embodiments, a target sequence is located in the nucleus or cytoplasm of a cell.

Thus, the CRISPR system comprises two distinct components, a guide RNA (gRNA) that hybridizes with the target sequence, and a nuclease (e.g. Type-II Cas9 protein), wherein the gRNA targets the target sequence and the nuclease (e.g. Cas9 protein) cleaves the target sequence. The guide RNA may comprise a combination of an endogenous bacterial crRNA and tracrRNA, i.e. the gRNA combines the targeting specificity of the crRNA with the scaffolding properties of the tracrRNA (required for Cas9 binding). Alternatively, the guide RNA may be a single guide RNA capable of directly binding Cas.

According to some embodiments of the invention, the editing agent (e.g., gRNAs) are specific to the target (Cp4.1LG13g07780) and don't have an off-target effect.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

In some embodiments, the tracr sequence has sufficient complementarity to a tracr mate sequence to hybridize and participate in formation of a CRISPR complex. As with the target sequence, a complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned.

Introducing CRISPR/Cas into a cell may be effected using one or more vectors driving expression of one or more elements of a CRISPR system such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. A single promoter may drive expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron).

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, transformed into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After transformation and positive selection, homologously targeted clones are identified. Next, a second targeting vector that contains a region of homology with the desired mutation is transformed into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Silencing at the Cp4.1LG13g07780 transcript (RNA) level can be effected using the below exemplary platforms, which are well known in the art.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene (Cp4.1LG13g07780). In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433. and Diallo et al, Oligonucleotides, Oct. 1, 2003, 13(5): 381-392. doi:10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the Cp4.1LG13g07780 gene (or its ortholog) mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Constructs useful in the methods according to the present invention may be constructed using recombinant DNA technology well known to persons skilled in the art. The coding sequence constructs may be inserted into vectors, which may be commercially available, suitable for transforming into plants and suitable for expression of the gene of interest in the transformed cells. The genetic construct can be an expression vector wherein the nucleic acid sequence is operably linked to one or more regulatory sequences allowing expression in the plant cells.

Plant cells may be transformed stably or transiently with the nucleic acid constructs of the present invention. Transient transformation can be done for instance in the case of genome editing to exclude the nuclease once editing is achieved (other ways to perform this were mentioned above).

In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:
(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.
(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by some embodiments of the invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

Molecular marker assisted selection (MAS) methods can be further employed in validating loss of function of the Cp4.1LG13g07780 gene or ortholog thereof following the genetic modification e.g., genome editing, breeding etc.

Thus, according to an aspect of the invention there is provided a method of identifying a Cucurbita pepo subsp. pepo plant useful for breeding e.g., crossing, the method comprising identifying in a genome of the plant at least one nucleic acid sequence alteration indicative of a multiflowering (mf) trait located between MF21 and MF26 of chromosome 13 using marker assisted selection (MAS), wherein identification of said at least one nucleic acid sequence variation is indicative of Cucurbita pepo subsp. pepo plant useful for crossing.

According to an aspect of the invention, there is provided a method of identifying a Cucurbita pepo subsp. pepo plant useful for crossing, the method comprising identifying in a genome of the plant at least one nucleic acid sequence alteration indicative of a multiflowering (mf) trait located between MF1 and MF2 of chromosome 13 using marker assisted selection (MAS), wherein identification of said at least one nucleic acid sequence variation is indicative of Cucurbita pepo subsp. pepo plant useful for crossing.

According to a specific embodiment, the loss of function mutation or at least one nucleic acid sequence alteration indicative of mf trait is located between MF29 and MF26 of chromosome 13.

Marker-assisted selection (MAS) involves the use of one or more of the molecular markers for the identification and selection of those progeny plants that contain one or more of the alleles that encode for the desired trait. MAS can be used to select progeny plants having the desired trait by identifying plants harboring the QTL(s) of interest, allowing for timely and accurate selection.

TABLE 4

| | | | | | polymorphism | | amplicon | amplicon |
|---|---|---|---|---|---|---|---|---|
| | primer F | location | primer R | location | type | location | in SET | in TRF |
| MF 1 | CTTCACATCTGCCCATTGTTC/SEQ ID | 6,634,240 | AAGACCAAGGCAAGAACACC/SEQ ID | 6,634,320 | INDEL 34 bp | 6,634,287 | 67 | 101 |
| | NO: 35 | | NO: 36 | | | | | |
| MF 2 | TCGTCAGGAGGGAAGAAAC/SEQ ID | 8,975,550 | AGGGGCTTCTCAGCGATTG/SEQ ID | 8,975,640 | INDEL 18 bp | 8,975,550 | 111 | 93 |
| | NO: 37 | | NO: 38 | | | | | |
| MF 3 | ACAAGCCGTAAGGATAGAGG/SEQ ID | 6,978,230 | AGAACCAACAAAATAGATCACC/SEQ | 6,978,350 | INDEL 25 bp | 6,978,300 | 153 | 129 |
| | NO: 39 | | ID NO: 40 | | | | | |
| MF 4 | CACATTCCTCCATAAAGTTC/SEQ ID | 7,290,100 | ATTTCAATTTCTCTCTTCTAC/SEQ ID | 7,290,000 | INDEL 37 bp | 7,290,080 | 80 | 117 |
| | NO: 41 | | NO: 42 | | | | | |
| MF 5 | TTAATTGCTATTTGAAGTATTGAG/SEQ | 8,513,100 | TCGTTCTAAATTACTTGCTATG/SEQ | 8,513,240 | INDEL 31 bp | 8,513,180 | 127 | 158 |
| | ID NO: 43 | | ID NO: 44 | | | | | |
| MF 6 | ATTTTCTCATTTATTTTACCAAC/SEQ | 8,498,680 | GCCATTGCCCCAAATTTCTT/SEQ ID | 8,498,780 | INDEL 19 bp | 8,498,720 | 101 | 120 |
| | ID NO: 45 | | NO: 46 | | | | | |
| MF 7 | GACACACAAAATCAAGAATCAA/SEQ | 8,004,200 | AAGGCCCATGGAGTGACC/SEQ ID | 8,004,360 | INDEL 100 | 8,004,280 | 70 | 170 |
| | ID NO: | | NO: | | bp | | | |
| MF 8 | CTAGCAAGAGGAGATGTCAC/SEQ ID | 8,027,450 | AGTTTGATTTTTGGGACTTACC/SEQ | 8,027,560 | INDEL 40 bp | 8,027,500 | 86 | 126 |
| | NO: | | ID NO: | | | | | |
| MF9 | AGATACACATTTTCAGTTTCAC/SEQ ID | 8,729,400 | CAAAGCTGCCCTAAAAACGG/SEQ ID | 8,729,470 | INDEL 17 bp | 8,729,434 | 107 | 90 |
| | NO: | | NO: | | | | | |
| MF | CCTTTGTGACACATTATTTAAG/SEQ ID | 8,212,690 | CATGTCTTGTTCGTTGTTCC/SEQ ID | 8,212,840 | INDEL 28 bp | 8,212,810 | 141 | 169 |
| 10 | NO: | | NO: | | | | | |
| MF | GTATGTATGTAATTAGCATTGTG/SEQ | 8,300,420 | AATCTGATGATGGCAACTTGC/SEQ ID | 8,300,500 | INDEL 20 bp | 8,300,450 | 119 | 99 |
| 11 | ID NO: | | NO: | | | | | |
| MF12 | GCGTTTTAAAACTTTGAGGGG/SEQ ID | 8,317,880 | CAAATGAGATCATCAACATACC/SEQ | 8,318,090 | INDEL 96 bp | 8,318,000 | 125 | 221 |
| | NO: | | ID NO: | | | | | |
| MF13 | CATAATTGGTGCTTATATTCAAC/SEQ | 8,599,210 | CTTCAACTTTGCCATCCCC/SEQ ID | 8,599,310 | INDEL 45 bp | 8,599,240 | 120 | 94 |
| | ID NO: | | NO: | | | | | |
| MF14 | CTTGCTCGTTATGCTGATTC/SEQ ID | 8,611,200 | ATGCTCAATCCACCATAAGG/SEQ ID | 8,611,340 | INDEL 26 bp | 8,611,260 | 104 | 149 |
| | NO: | | NO: | | | | | |
| MF15 | TCCATACAGTAGTGCTAAGG/SEQ ID | 8,616,735 | CTGCTTTATGATTGAATCTATG/SEQ | 8,616,840 | INDEL 16 bp | 8,616,770 | 105 | 121 |
| | NO: | | ID NO: | | | | | |
| MF16 | TCAAGCCTGCAAGATGTAGC/SEQ ID | 8,152,210 | GCATTCCACACCCTTATAAAG/SEQ ID | 8,152,620 | INDEL 230 | 8,152,400 | 200 | 432 |
| | NO: | | NO: | | bp | | | |
| MF17 | TTCTGAAGATTTATGTTTTGCC/SEQ ID | 8,172,960 | TCTGTTCTAAAAGCCATTCTC/SEQ ID | 8,173,130 | INDEL 53 bp | 8,173,030 | 138 | 191 |
| | NO: | | NO: | | | | | |
| MF18 | TCACAATAGAAGTCATTTAATAC/SEQ | 8,413,060 | CCATTTTGGTCTCTTGCTTTC/SEQ ID | 8,413,235 | INDEL 65 bp | 8,413,150 | 130 | 195 |
| | ID NO: | | NO: | | | | | |
| MF19 | CCATTTTGGTCCCTATTTACA/SEQ ID | 8,413,020 | CCATTTTGGTCTCTTGCTTTC/SEQ ID | 8,413,235 | INDEL 35 bp | 8,413,150 | 170 | 235 |
| | NO: | | NO: | | | | | |
| MF20 | GCACTGTGTATGGACTTCTC/SEQ ID | 8,344,950 | GATTCTCAATGATTAAAAACATG/SEQ | 8,345,080 | INDEL 65 bp | 8,345,030 | 110 | 144 |
| | NO: | | ID NO: | | | | | |
| MF21 | GTTTCATTAAAAGTAGTCTAAGG/SEQ | 8,361,040 | TTGACCTTACTGGCACACG/SEQ ID | 8,361,230 | INDEL 21 bp | 8,361,200 | 176 | 201 |
| | ID NO: | | NO: | | | | | |
| MF22 | TATGTGCGCAGACCTATTTTC/SEQ ID | 8,385,240 | ATGTTCTTCGTAGTTCTGTTC/SEQ ID | 8,385,380 | INDEL 25 bp | 8,385,330 | 142 | 163 |
| | NO: | | NO: | | | | | |
| MF23 | GTTCCCAGCCATCATTTTCC/SEQ ID | 8,333,620 | TCAGCACATTCACGTCCAAC/SEQ ID | 8,333,690 | INDEL 12 bp | 8,333,644 | 112 | 90 |
| | NO: | | NO: | | | | | |
| MF24 | CATGGAAGCTTACTACACAG/SEQ ID | 8,373,100 | CTGTTGTTAGATTCTTAAGATG/SEQ | 8,373,210 | INDEL 18 bp | 8,373,139 | 109 | 127 |
| | NO: | | ID NO: | | | | | |
| MF25 | GTGTTATTACCAGGATGGATC/SEQ ID | 8,325,630 | CTGAACTGTACTTCGAGTCG/SEQ ID | 8,325,710 | INDEL 22 bp | 8,325,660 | 84 | 106 |
| | NO: | | NO: | | | | | |
| MF26 | CTAGCAGTCTCACAGGGAG/SEQ ID | 8,367,570 | TGTTCTCCTCCCCAACCAG/SEQ ID | 8,367,840 | INDEL 115 | 8,367,700 | 170 | 285 |
| | NO: | | NO: | | bp | | | |
| MF27 | TATGATTAATGGAAAGTTGATTTG/SEQ | 8,370,040 | GTTCGAGGTTAATCTTGAATC/SEQ ID | 8,370,130 | INDEL 22 bp | 8,370,100 | 89 | 111 |
| | ID NO: | | NO: | | | | | |
| MF28 | GGCATCACAAAATTAAAATGTAG/SEQ | 8,327,100 | CATAGCCCCAACCCAACC/SEQ ID NO: | 8,327,180 | INDEL 11 bp | 8,327,165 | 88 | 97 |
| | ID NO: | | | | | | | |
| MF29 | ACAAACCCACCGCTACTAG/SEQ ID | 8,363,110 | GTATCAGAGCCAGTCACTG/SEQ ID | 8,363,280 | INDEL 10 bp | 8,363,191 | 198 | 188 |
| | NO: | | NO: | | | | | |

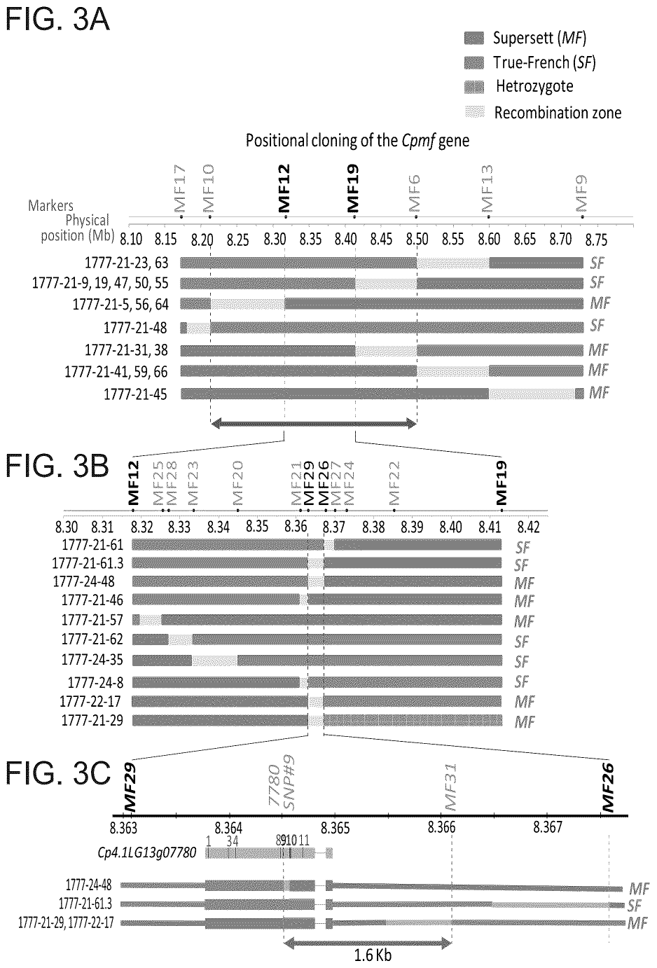

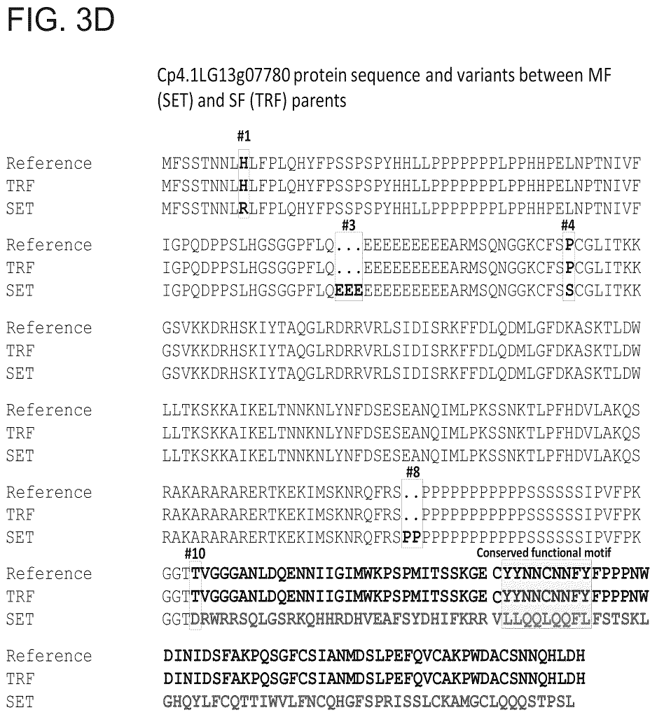

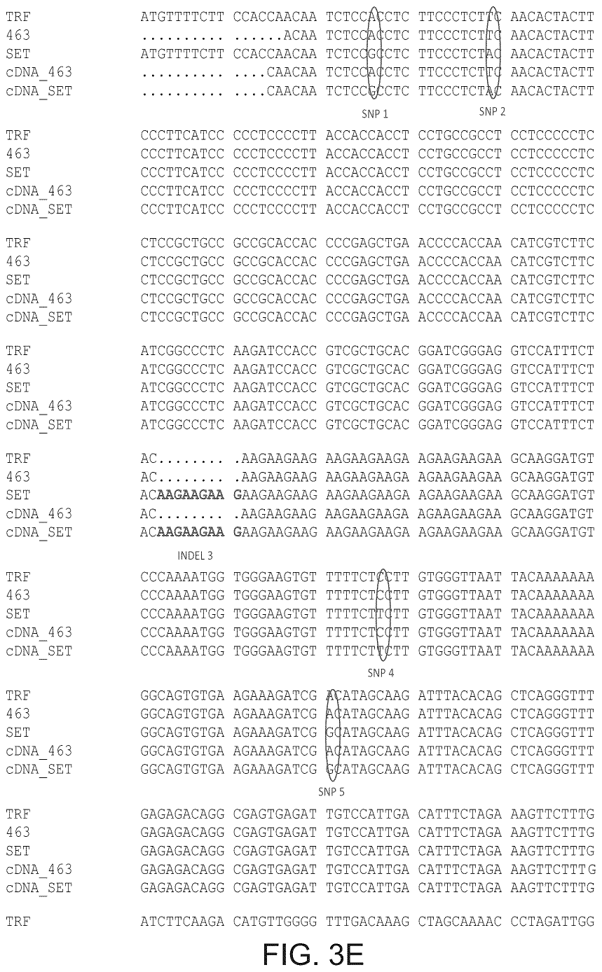

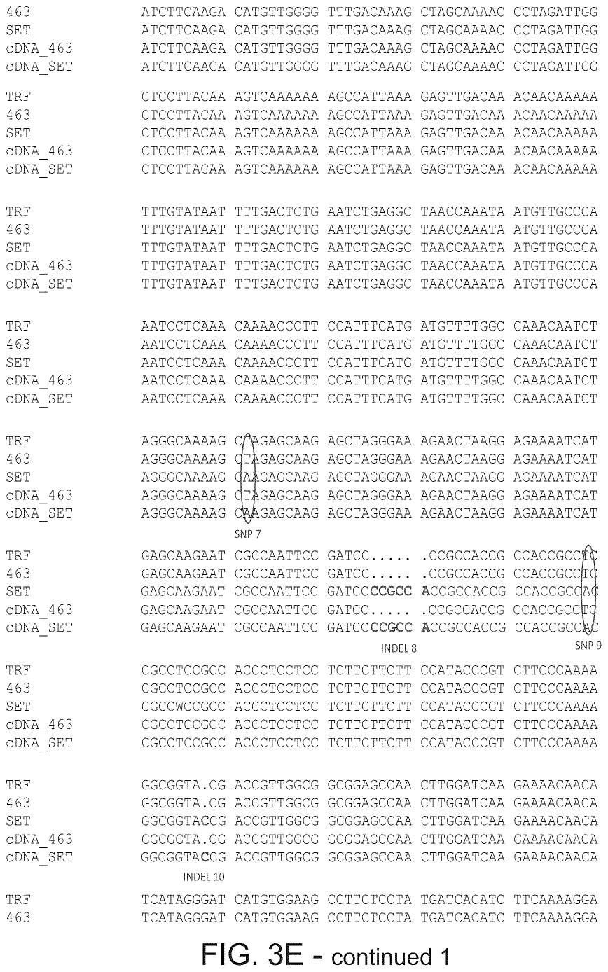

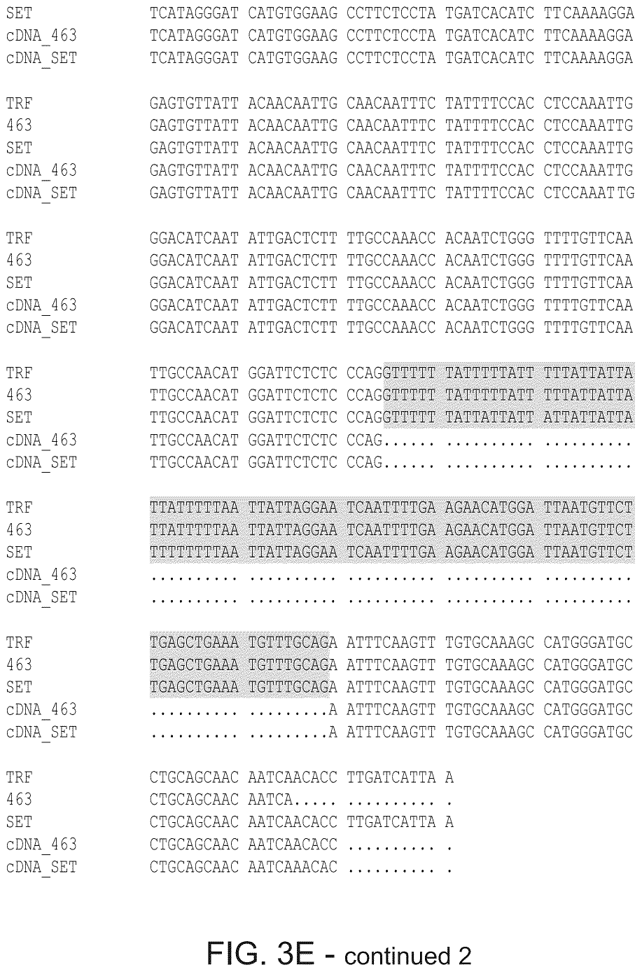

FIGS. 3E and 4 show sequence variations which can be used in MAS.

According to a specific embodiment, said loss of function mutation or at least one nucleic acid sequence alteration indicative of mf trait is located between MF21 and MF26 of chromosome 13 or any marker therebetween.

According to a specific embodiment, said loss of function mutation or at least one nucleic acid sequence alteration indicative of mf trait is located between MF29 and MF26 of chromosome 13 or any marker therebetween.

The assessment is typically done at the nucleic acid level though identification of the loss-of function mutation/sequence alteration can be determined at the protein level such as by using an immunoassay (e.g., Western blot).

Specific primers or probes used for MAS according to the present teachings can be packed in a kit which has instructions for executing the method of identification of mf as described herein.

Alternatively or additionally, the validation is performed using phenotypic methods such as described herein (e.g., total yield, cumulative number of fruit/flower per plant or per plot in a growing season, bud/flower per leaf axil and/or tendril architecture).

Under its normal function TCP transcription factor is regulating the normal development of tendril structure. According to a specific embodiment, the mutation is causing the loss of this function resulting in the loss of common tendril identity towards an abnormal leaf-like structure.

Alternatively or additionally, the validation is performed using biochemical methods such as testing protein-DNA binding, transcriptional assays and other which are known to determine the activity of transcription factors.

The present teachings further relate to plant parts which comprise the loss of function mutation.

These parts include, but are not limited to, leaves, seeds, shoots, fruits, flowers, stems, roots, and plant cells or tissues. The plant may be in any form including suspension cultures, embryos, meristematic regions, callus tissue, leaves, gametophytes, sporophytes, pollen, and microspores.

According to a specific embodiment, the plant part is a fruit.

According to a specific embodiment, the plant part is a cutting.

According to a specific embodiment, the plant part is a seed.

Embodiments described herein also relate to the germplasm of the plants. The germplasm is constituted by all inherited characteristics of an organism.

Once the plants are at hand they can be used in further breeding programs.

Thus, there is provided a method of growing the plant, as described herein.

Also provided herein are nucleic acid agents (e.g., primers or probes) for performing MAS or for identifying the plants or plant parts of the invention. These are typically designed to specifically hybridize with the genomic sequence to detect the nucleic acid alteration or loss of function mutation. Some examples are provided in Table 4.

Alternatively or additionally they can be used in the Cucurbita pepo subsp. pepo industry for any practical application.

Thus, according to an aspect of the invention there is provided a method of producing an edible product, comprising processing the fruit or flower of the plant as described herein (comprising the loss of function mutation in the gene or ortholog).

According to a specific embodiment, the product comprises DNA of the plant.

According to a specific embodiment, the product does not comprises DNA of the plant.

The processing can include any food related processing method such as pressing, drying, frying, cooking, extracting, grating, shredding, sieving, filtering, freezing, cooling and more.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Genetic Mapping of the Mf Trait

To map the mf gene, the present inventors used two advanced backcross populations at the BC6F2 generation. The populations and their development are described in Paris and Gur Euphytica volume 218, Article number: 19 (2022). The advanced backcross population of Zucchini was derived as described in Paris and Hanan (2010), specifically: A plant of Cucurbita pepo subsp. pepo Cocozelle Group ‘Striato Pugliese’ (Ingegnoli, Milan, Italy), which was single-flowering, was crossed with a plant from ‘Supersett’ of C. pepo subsp. ovifera Crookneck Group, which is multiple-flowering. An F1 plant was self-pollinated and a resulting multiple-flowering F2 plant was selected and crossed with a plant of a highly inbred line derived from the single-flowering C. pepo subsp. pepo Zucchini Group ‘True French’; an F1 plant was then self-pollinated and a resulting multiple-flowering F2 plant was selected and back-crossed to the ‘True French’ inbred to obtain first backcross-generation (BC1) plants. The cycle of selecting multiple-flowering plants for backcrossing and self-pollination of plants resulting from backcrossing to the ‘True French’ inbred as the recurrent parent, was continued through the sixth back-cross generation. Accession 1777, a true-breeding, multiple-flowering derivative from the sixth back-cross generation, therefore can be considered as nearly isogenic with ‘True French’, but, unlike that cultivar, its plants bear two flowers at some stem nodes. Plants of Accession 1777 and ‘True French’ were intercrossed, and reciprocal F1 plants were self-pollinated to form the experimental population.

The advanced backcross population of Cocozelle was derived similarly, by the same initial cross of the subsp. pepo Cocozelle ‘Striato Pugliese’ and the subsp. ovifera Crookneck ‘Supersett’, with the F1 self-pollinated and a resulting multiple-flowering F2 plant selected for crossing on Cocozelle ‘Inbred 463-7-6-15’, a derivative of the Cocozelle ‘Striato d'Italia’. As described briefly by Paris and Gur (2022), the cycle of selecting multiple-flowering plants for backcrossing, and self-pollination of plants resulting from the backcrossing to the Cocozelle ‘Inbred 463-7-6-15’, was continued through the sixth back-cross generation. Accession 1951, a true-breeding, multiple-flowering derivative from the sixth back-cross generation, therefore can be considered as nearly isogenic with Inbred 463-7-6-15 but, unlike Inbred 463-7-6-15, its plants bear two or more flowers at some stem nodes. Plants of Accession 1951 and ‘Inbred 463-7-6-15’ were intercrossed, and reciprocal F1 plants were self-pollinated to form the experimental population.

From each of these experimental populations, the present inventors grew during Summer 2021~120 nearly isogenic segregants that were characterized for the single- or multiple-flowering phenotype. From each population ~25 plants were selected that were single-flowering (Mf/−) and 25 plants that were multiple-flowering (mf/mf) and they were used for Bulk-Sequencing analysis (BSA-Seq). High-quality genomic DNA was extracted from each of these plants. The Zucchini samples were used to create separate DNA bulks from the Mf and mf plants, and these bulks alongside the population parental lines (the Zucchini ‘True French’ (Mf/Mf) and the Crookneck ‘Supersett’ (mf/mf)) were subjected to whole-genome resequencing. The sequencing resulted in more than 700,000 informative SNPs that distinguish between the parental lines. The present inventors then performed genome-wide Delta-SNP-Index analysis using allele frequencies values and found a single locus on chromosome 13 (~2 Mbp interval, at 7.0-9.0 Mbp) associated with the multiple-flowering trait.

Following the initial mapping of the mf gene to a 2 Mbp interval on Cucurbita pepo chromosome 13, the present inventors started to zoom in on this region to fine-map the trait. PCR markers were developed in this target region and used initially to screen the individual plants that composed the DNA bulks for validation and identification of recombinants in the region. The analysis was also expanded to the Cocozelle ‘Inbred 463-7-6-15’ BC6F2 population to validate the mapping results and identify more recombinations. This analysis resulted in narrowing the interval to a common confidence interval of ~550 Kbp (8.173-8.729b Mbp). This interval contains 80 putative genes.

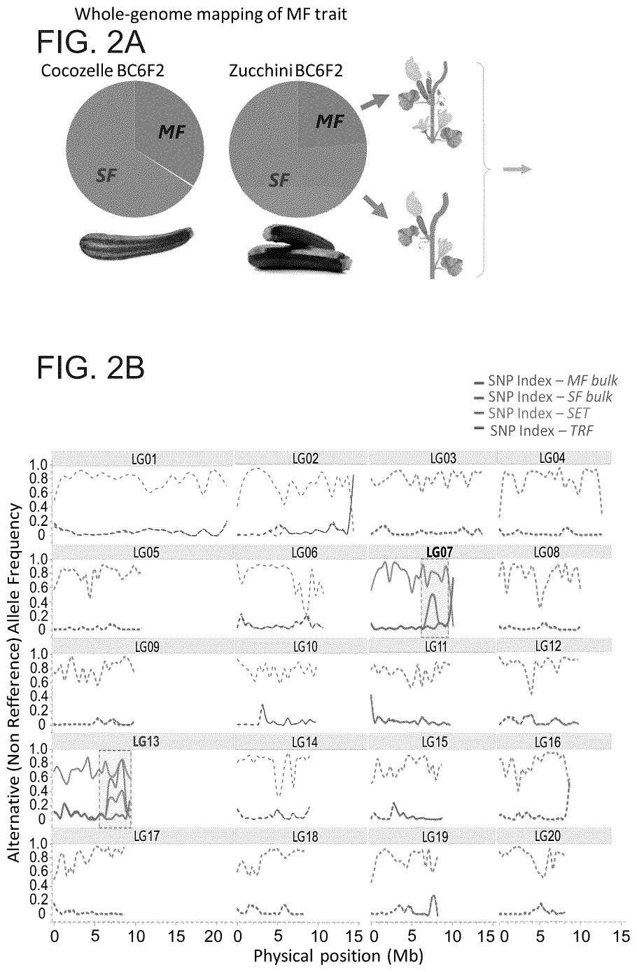

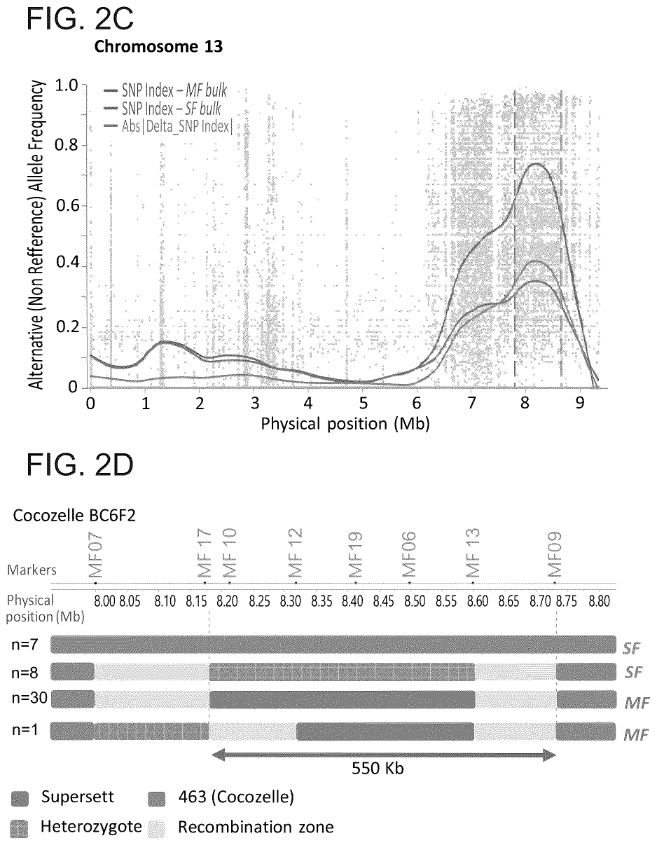

Next step was fine-mapping through substitution mapping approach using additional recombinants in the target region. Two rounds of fine-mapping were performed to narrow the interval to the causative gene resolution. ~2,000 BC6F2 seeds were genotyped using flanking markers at the trait interval to identify new recombinants (markers list and information provided in Table 4 above). The recombinant plants were grown and phenotyped for their flowering pattern (MF or SF) and their precise genotype at the target interval was defined using additional internal PCR markers (listed in Table 4). Relevant BC6F2 recombinants were self-polinated to obtain BC6F3 seeds and progeny tests as well as recombination fixation was performed. As a result of this detailed and extensive genetic mapping analysis, the trait region was defined to the very narrow physical interval 8,361,200-8,367,700 Mb on chromosome 13 of the Zucchini genome (Cucurbita pepo (MU-CU-16) v4.1 www(dot)cucurbitgenomics(dot)org/v2/organism/4, see FIGS. 2A-D and 3A-B). using additional recombinants this was further narrowed down to a minimal 1,600 bp interval using the flanking markers 7780 SNP #9 and MF31, as shown in FIG. 3C. A single annotated gene is found within this narrow genomic interval, Cp4.1LG13g07780, annotated as TCP Transcription-factor DICHOTOMA-like (www(dot)cucurbitgenomics(dot)org/v2/feature/gene/Cp4.1LG13g07780). Several significant non-synonymous sequence polymorphisms were identified within this gene differentiating between the mapping population parents and between additional single- and multiple-flowering C. pepo accessions. Most prominent is a single-base insertion causing frame-shift in translation followed by substantial change in the predicted sequence of this TCP protein (Also referred to herein as SNP #10, See FIGS. 3D, 3E and 4).

SNP #5, A/G polymorphism, located at position 8,364,142 in the first exon of the Cp4.1LG13g07780 gene was used for development of CAPs marker for MAS of the MF allele.

Primer forward: 07780#5 F
(SEQ ID NO: 32)
CACATTACAAACCTAAAGACC
position: 8,363,790
Primer reverse: 07780#5 R
(SEQ ID NO: 33)
TCTTTCCCTAGCTCTTGCTC
position: 8,364,440

Product size 676 bp. Polymorphism for restriction with Taq1 is caused by this SNP. SET allele is digested and TRF not. Restriction products are 360 and 361 bps

Product sequence is shown below (SEQ ID NO: 34). Primers are in bold and underline.

CACATTACAAACCTAAAGACCCTAAACTTACCCCAAAACCACACAAAAAA
ACATGTTTTCTTCCACCAACAATCTCCACCTCTTCCCTCTTCAACACTAC
TTCCCTTCATCCCCCTCCCCTTACCACCACCTCCTGCCGCCTCCTCCCCC
TCCTCCGCTGCCGCCGCACCACCCCGAGCTGAACCCCACCAACATCGTCT
TCATCGGCCCTCAAGATCCACCGTCGCTGCACGGATCGGGAGGTCCATTT
CTACAAGAAGAAGAAGAAGAAGAAGAAGAAGAAGCAAGGATGTCCCAAAA
TGGTGGGAAGTGTTTTTCTCCTTGTGGGTTAATTACAAAAAAAGGCAGTG
TGAAGAAAGATCGACATAGCAAGATTTACACAGCTCAGGGTTTGAGAGAC
AGGCGAGTGAGATTGTCCATTGACATTTCTAGAAAGTTCTTTGATCTTCA
AGACATGTTGGGGTTTGACAAAGCTAGCAAAACCCTAGATTGGCTCCTTA
CAAAGTCAAAAAAAGCCATTAAAGAGTTGACAAACAACAAAAATTTGTAT
AATTTTGACTCTGAATCTGAGGCTAACCAAATAATGTTGCCCAAATCCTC
AAACAAAACCCTTCCATTTCATGATGTTTTGGCCAAACAATCTAGGGCAA
AAGCTAGAGCAAGAGCTAGGGAAAGA

FIG. 4 shows a gel image of CAPs marker developed for SNP #5 in the Cp4.1LG13g07780 gene.

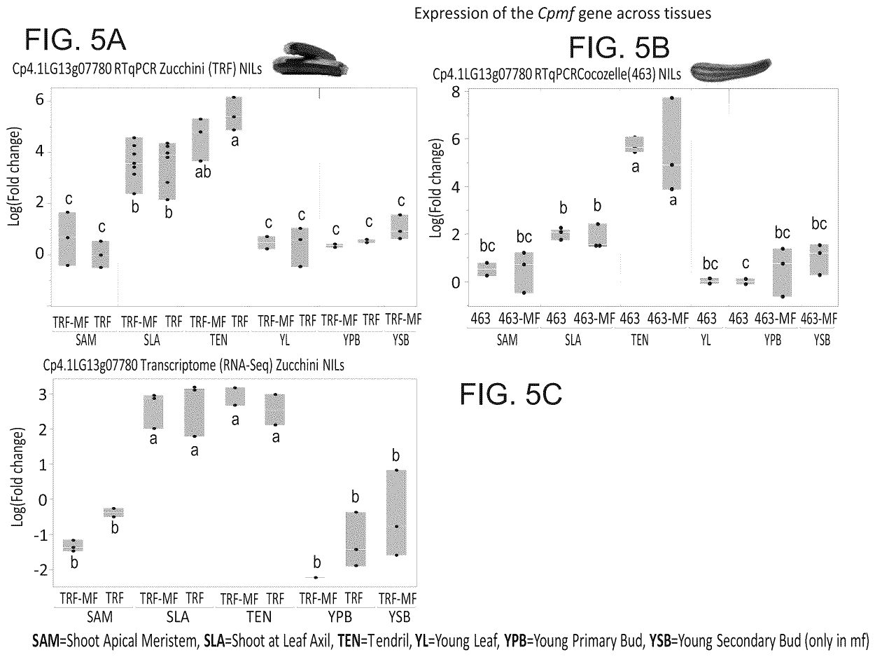

FIGS. 5A-C shows that Cpmf gene is not differentially expressed between MF NILs, and is expressed specifically in tendrils and at the stem at leaf axil. (a) Comparison of expression of Cp4.1LG13g07780 (by qRT-PCR) between Zucchinni MF NILs across six tissues. (b) Comparison of expression of Cp4.1LG13g07780 (by qRT-PCR) between Cocozelle MF NILs across six tissues. (c) Comparison of expression of Cp4.1LG13g07780 (by RNA-Seq) between Zucchinni MF NILs across five tissues. Different letters indicate significant difference of the means at P<0.05.

In consensus with the dual function of the Cpmf TCP gene the present inventors show that it is exclusively expressed in tendrils and at the stem at leaf axil, both are the target tissues where the phenotypic impact of the mutation is observed. In both melon and cucumber the homolog gens are reported as tendril-specific.

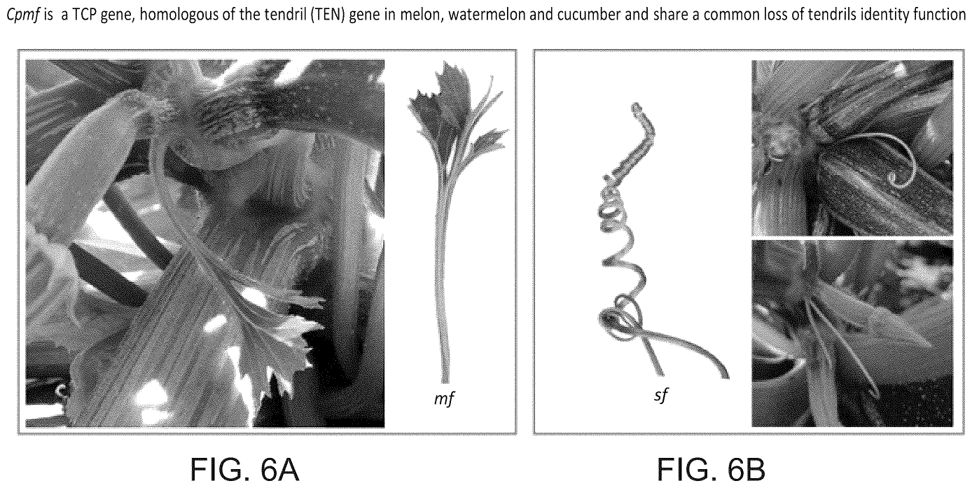

FIGS. 6A-B show that: Cpmf is a TCP gene, homolog of the tendril (TEN) gene in melon and cucumber and share a common loss of tendrils identity regulation function. (a) Modified leaf-like tendril in the Zucchinni (TRF) MF nearly-isogenic line. (b) Normal tendril in the Zucchinni (TRF) SF nearly-isogenic line.

In this figure the phenotypic impact of the Cpmf mutation on tendril development is demonstrated, such that there is a loss of normal tendril development and instead development of leaf-like structure of the tendril. This phenotype is common to mutations in the homolog genes in melon and cucumber.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A Cucurbita pepo subsp. pepo plant comprising a genome having a loss of function mutation in a Cp4.1LG13g07780 gene or ortholog thereof, wherein the loss of function mutation confers a multiflowering (mf) trait and wherein when the loss of function mutation is in an introgression from a Cucurbita pepo subsp. ovifera var. Crookneck ‘Supersett’, said introgression is smaller than 700 kb or wherein the plant is not of a variety, seeds of which, having been deposited at NCIMB Ltd. under Accession No. NCIMB 41744 (herein referred to as Multizuq) or progeny thereof or Accession No. NCIMB 41794 (herein referred to as Nizzan) or progeny thereof.

2. A method of producing a Cucurbita pepo subsp. pepo plant having a multiflowering (mf) trait, the method comprising down-regulating a Cp4.1LG13g07780 gene or ortholog thereof in the Cucurbita pepo subsp. pepo plant to thereby produce a Cucurbita pepo subsp. pepo plant having the mf trait, and wherein when said down-regulating is by crossing with a Cucurbita pepo subsp. ovifera:
an introgression resultant of said crossing is not that present in seeds having been deposited at NCIMB Ltd. under Accession No. NCIMB 41744 (herein referred to as Multizuq) or progeny thereof or Accession No. NCIMB 41794 (herein referred to as Nizzan) or progeny thereof; or
an introgression resultant of said crossing which comprises said ortholog of Cp4.1LG13g07780 is smaller than 700 kb.

3. A method of identifying a Cucurbita pepo subsp. pepo plant useful for crossing, the method comprising identifying in a genome of the plant at least one nucleic acid sequence alteration indicative of a multiflowering (mf) trait located between MF1 and MF2 or MF21 and MF26 of chromosome 13 using marker assisted selection (MAS), wherein identification of said at least one nucleic acid sequence variation is indicative of Cucurbita pepo subsp. pepo plant useful for crossing.

4. A method of processing a Cucurbita pepo subsp. pepo plant, the method comprising subjecting the plant of claim 1 to a process selected from the group consisting of cooking, baking, drying, extracting and frying thereby processing the Cucurbita pepo subsp. pepo plant.

5. The plant of claim 1, wherein said at least one loss of function mutation is in a homozygous form.

6. The method of claim 2, wherein said down-regulating is by genome editing.

7. The method of claim 2, wherein said down-regulating is by RNA silencing.

8. The method of claim 2, wherein said down-regulating is by breeding with a plant of a Cucurbita pepo subsp. ovifera and backcrossing with the Cucurbita pepo subsp. pepo plant.

9. The method of claim 2, wherein said down-regulating results in a loss of function mutation in said Cp4.1LG13g07780 gene or ortholog thereof.

10. The method of claim 3, further comprising validating presence of said loss-of-function mutation in said Cp4.1LG13g07780 gene or ortholog thereof.

11. The method of claim 10, wherein said validating is effected using molecular markers.

12. The plant of claim 1, wherein said loss of function mutation or at least one nucleic acid sequence alteration is in Exon 1 of said Cp4.1LG13g07780 gene or ortholog thereof.

13. The plant of claim 1, wherein said loss of function mutation or at least one nucleic acid sequence alteration is in a YNNCNNFY sequence encoded by the gene or ortholog thereof.

14. The plant of claim 1, wherein said loss of function mutation or at least one nucleic acid sequence alteration is within Exon 1 of the gene or a corresponding position of an ortholog thereof.

15. The plant of claim 1, wherein said loss of function mutation or at least one nucleic acid sequence alteration at position 8,364,572 within Exon 1 of the gene or a corresponding position of an ortholog thereof is a frameshift mutation.

16. The plant of claim 1, wherein said Cucurbita pepo subsp. ovifera is of Group Crookneck.

17. The plant of claim 1, wherein said Cucurbita pepo subsp. pepo plant is of Group Zucchini or Cocozelle.

18. The plant of claim 1, wherein said loss of function mutation or at least one nucleic acid sequence alteration indicative of mf trait is located between MF21 and MF26 of chromosome 13.

19. The plant of claim 1, wherein said loss of function mutation or at least one nucleic acid sequence alteration indicative of mf trait is located between MF29 and MF26 of chromosome 13.

20. The plant of claim 1, wherein said loss of function mutation causes loss of tendrils identity regulation function.


SEQUENCE LISTING (74 sequences)

SEQ ID NO: 1 - DNA, 1115 bp, Cucurbita pepo
  source 1..1115: mol_type = genomic DNA
atgttttcttccaccaacaatctccacctcttccctcttcaacactacttcccttcatccccctccccttaccaccacctcctgccgcctcctccccctcctccgctgccgccgcaccaccccgagctgaaccccaccaacatcgtcttcatcggccctcaagatccaccgtcgctgcacggatcgggaggtccatttctacaagaagaagaagaagaagaagaagaagaagcaaggatgtcccaaaatggtgggaagtgtttttctccttgtgggttaattacaaaaaaaggcagtgtgaagaaagatcgacatagcaagatttacacagctcagggtttgagagacaggcgagtgagattgtccattgacatttctagaaagttctttgatcttcaagacatgttggggtttgacaaagctagcaaaaccctagattggctccttacaaagtcaaaaaaagccattaaagagttgacaaacaacaaaaatttgtataattttgactctgaatctgaggctaaccaaataatgttgcccaaatcctcaaacaaaacccttccatttcatgatgttttggccaaacaatctagggcaaaagctagagcaagagctagggaaagaactaaggagaaaatcatgagcaagaatcgccaattccgatccccgccaccgccaccgcctccgcctccgccaccctcctcctcttcttcttccatacccgtcttcccaaaaggcggtacgaccgttggcggcggagccaacttggatcaagaaaacaacatcatagggatcatgtggaagccttctcctatgatcacatcttcaaaaggagagtgttattacaacaattgcaacaatttctattttccacctccaaattgggacatcaatattgactcttttgccaaaccacaatctgggttttgttcaattgccaacatggattctctcccaggttttttatttttatttttattattattatttttaattattaggaatcaattttgaagaacatggattaatgttcttgagctgaaatgtttgcagaatttcaagtttgtgcaaagccatgggatgcctgcagcaacaatcaacaccttgatcattaa

SEQ ID NO: 2 - RNA, 1020 nt, Cucurbita pepo
  source 1..1020: mol_type = mRNA
atgttttcttccaccaacaatctccacctcttccctcttcaacactacttcccttcatccccctccccttaccaccacctcctgccgcctcctccccctcctccgctgccgccgcaccaccccgagctgaaccccaccaacatcgtcttcatcggccctcaagatccaccgtcgctgcacggatcgggaggtccatttctacaagaagaagaagaagaagaagaagaagaagcaaggatgtcccaaaatggtgggaagtgtttttctccttgtgggttaattacaaaaaaaggcagtgtgaagaaagatcgacatagcaagatttacacagctcagggtttgagagacaggcgagtgagattgtccattgacatttctagaaagttctttgatcttcaagacatgttggggtttgacaaagctagcaaaaccctagattggctccttacaaagtcaaaaaaagccattaaagagttgacaaacaacaaaaatttgtataattttgactctgaatctgaggctaaccaaataatgttgcccaaatcctcaaacaaaacccttccatttcatgatgttttggccaaacaatctagggcaaaagctagagcaagagctagggaaagaactaaggagaaaatcatgagcaagaatcgccaattccgatccccgccaccgccaccgcctccgcctccgccaccctcctcctcttcttcttccatacccgtcttcccaaaaggcggtacgaccgttggcggcggagccaacttggatcaagaaaacaacatcatagggatcatgtggaagccttctcctatgatcacatcttcaaaaggagagtgttattacaacaattgcaacaatttctattttccacctccaaattgggacatcaatattgactcttttgccaaaccacaatctgggttttgttcaattgccaacatggattctctcccagaatttcaagtttgtgcaaagccatgggatgcctgcagcaacaatcaacaccttgatcattaa

SEQ ID NO: 3 - DNA, 1020 bp, Cucurbita pepo
  source 1..1020: mol_type = genomic DNA
atgttttcttccaccaacaatctccacctcttccctcttcaacactacttcccttcatccccctccccttaccaccacctcctgccgcctcctccccctcctccgctgccgccgcaccaccccgagctgaaccccaccaacatcgtcttcatcggccctcaagatccaccgtcgctgcacggatcgggaggtccatttctacaagaagaagaagaagaagaagaagaagaagcaaggatgtcccaaaatggtgggaagtgtttttctccttgtgggttaattacaaaaaaaggcagtgtgaagaaagatcgacatagcaagatttacacagctcagggtttgagagacaggcgagtgagattgtccattgacatttctagaaagttctttgatcttcaagacatgttggggtttgacaaagctagcaaaaccctagattggctccttacaaagtcaaaaaaagccattaaagagttgacaaacaacaaaaatttgtataattttgactctgaatctgaggctaaccaaataatgttgcccaaatcctcaaacaaaacccttccatttcatgatgttttggccaaacaatctagggcaaaagctagagcaagagctagggaaagaactaaggagaaaatcatgagcaagaatcgccaattccgatccccgccaccgccaccgcctccgcctccgccaccctcctcctcttcttcttccatacccgtcttcccaaaaggcggtacgaccgttggcggcggagccaacttggatcaagaaaacaacatcatagggatcatgtggaagccttctcctatgatcacatcttcaaaaggagagtgttattacaacaattgcaacaatttctattttccacctccaaattgggacatcaatattgactcttttgccaaaccacaatctgggttttgttcaattgccaacatggattctctcccagaatttcaagtttgtgcaaagccatgggatgcctgcagcaacaatcaacaccttgatcattaa

SEQ ID NO: 4 - protein, 339 aa, Cucurbita pepo
MFSSTNNLHLFPLQHYFPSSPSPYHHLLPPPPPPPLPPHHPELNPTNIVFIGPQDPPSLHGSGGPFLQEEEEEEEEEARMSQNGGKCFSPCGLITKKGSVKKDRHSKIYTAQGLRDRRVRLSIDISRKFFDLQDMLGFDKASKTLDWLLTKSKKAIKELTNNKNLYNFDSESEANQIMLPKSSNKTLPFHDVLAKQSRAKARARARERTKEKIMSKNRQFRSPPPPPPPPPPPSSSSSSIPVFPKGGTTVGGGANLDQENNIIGIMWKPSPMITSSKGECYYNNCNNFYFPPPNWDINIDSFAKPQSGFCSIANMDSLPEFQVCAKPWDACSNNQHLDH

SEQ ID NO: 5 - DNA, 1115 bp, synthetic construct; identical to SEQ ID NO: 1
  source 1..1115: mol_type = other DNA

SEQ ID NO: 6 - DNA, 1083 bp, synthetic construct
  source 1..1083: mol_type = other DNA
acaatctccacctcttccctcttcaacactacttcccttcatccccctccccttaccaccacctcctgccgcctcctccccctcctccgctgccgccgcaccaccccgagctgaaccccaccaacatcgtcttcatcggccctcaagatccaccgtcgctgcacggatcgggaggtccatttctacaagaagaagaagaagaagaagaagaagaagcaaggatgtcccaaaatggtgggaagtgtttttctccttgtgggttaattacaaaaaaaggcagtgtgaagaaagatcgacatagcaagatttacacagctcagggtttgagagacaggcgagtgagattgtccattgacatttctagaaagttctttgatcttcaagacatgttggggtttgacaaagctagcaaaaccctagattggctccttacaaagtcaaaaaaagccattaaagagttgacaaacaacaaaaatttgtataattttgactctgaatctgaggctaaccaaataatgttgcccaaatcctcaaacaaaacccttccatttcatgatgttttggccaaacaatctagggcaaaagctagagcaagagctagggaaagaactaaggagaaaatcatgagcaagaatcgccaattccgatccccgccaccgccaccgcctccgcctccgccaccctcctcctcttcttcttccatacccgtcttcccaaaaggcggtacgaccgttggcggcggagccaacttggatcaagaaaacaacatcatagggatcatgtggaagccttctcctatgatcacatcttcaaaaggagagtgttattacaacaattgcaacaatttctattttccacctccaaattgggacatcaatattgactcttttgccaaaccacaatctgggttttgttcaattgccaacatggattctctcccaggttttttatttttatttttattattattatttttaattattaggaatcaattttgaagaacatggattaatgttcttgagctgaaatgtttgcagaatttcaagtttgtgcaaagccatgggatgcctgcagcaacaatca

SEQ ID NO: 7 - DNA, 1131 bp, synthetic construct
  source 1..1131: mol_type = other DNA
atgttttcttccaccaacaatctccgcctcttccctctacaacactacttcccttcatccccctccccttaccaccacctcctgccgcctcctccccctcctccgctgccgccgcaccaccccgagctgaaccccaccaacatcgtcttcatcggccctcaagatccaccgtcgctgcacggatcgggaggtccatttctacaagaagaagaagaagaagaagaagaagaagaagaagaagcaaggatgtcccaaaatggtgggaagtgtttttcttcttgtgggttaattacaaaaaaaggcagtgtgaagaaagatcggcatagcaagatttacacagctcagggtttgagagacaggcgagtgagattgtccattgacatttctagaaagttctttgatcttcaagacatgttggggtttgacaaagctagcaaaaccctagattggctccttacaaagtcaaaaaaagccattaaagagttgacaaacaacaaaaatttgtataattttgactctgaatctgaggctaaccaaataatgttgcccaaatcctcaaacaaaacccttccatttcatgatgttttggccaaacaatctagggcaaaagcaagagcaagagctagggaaagaactaaggagaaaatcatgagcaagaatcgccaattccgatccccgccaccgccaccgccaccgccaccgccwccgccaccctcctcctcttcttcttccatacccgtcttcccaaaaggcggtaccgaccgttggcggcggagccaacttggatcaagaaaacaacatcatagggatcatgtggaagccttctcctatgatcacatcttcaaaaggagagtgttattacaacaattgcaacaatttctattttccacctccaaattgggacatcaatattgactcttttgccaaaccacaatctgggttttgttcaattgccaacatggattctctcccaggttttttattattattattattattattttttttaattattaggaatcaattttgaagaacatggattaatgttcttgagctgaaatgtttgcagaatttcaagtttgtgcaaagccatgggatgcctgcagcaacaatcaacaccttgatcattaa

SEQ ID NO: 8 - DNA, 995 bp, synthetic construct
  source 1..995: mol_type = other DNA
caacaatctccacctcttccctcttcaacactacttcccttcatccccctccccttaccaccacctcctgccgcctcctccccctcctccgctgccgccgcaccaccccgagctgaaccccaccaacatcgtcttcatcggccctcaagatccaccgtcgctgcacggatcgggaggtccatttctacaagaagaagaagaagaagaagaagaagaagcaaggatgtcccaaaatggtgggaagtgtttttctccttgtgggttaattacaaaaaaaggcagtgtgaagaaagatcgacatagcaagatttacacagctcagggtttgagagacaggcgagtgagattgtccattgacatttctagaaagttctttgatcttcaagacatgttggggtttgacaaagctagcaaaaccctagattggctccttacaaagtcaaaaaaagccattaaagagttgacaaacaacaaaaatttgtataattttgactctgaatctgaggctaaccaaataatgttgcccaaatcctcaaacaaaacccttccatttcatgatgttttggccaaacaatctagggcaaaagctagagcaagagctagggaaagaactaaggagaaaatcatgagcaagaatcgccaattccgatccccgccaccgccaccgcctccgcctccgccaccctcctcctcttcttcttccatacccgtcttcccaaaaggcggtacgaccgttggcggcggagccaacttggatcaagaaaacaacatcatagggatcatgtggaagccttctcctatgatcacatcttcaaaaggagagtgttattacaacaattgcaacaatttctattttccacctccaaattgggacatcaatattgactcttttgccaaaccacaatctgggttttgttcaattgccaacatggattctctcccagaatttcaagtttgtgcaaagccatgggatgcctgcagcaacaatcaacacc

SEQ ID NO: 9 - DNA, 980 bp, synthetic construct
  source 1..980: mol_type = other DNA
caacaatctccgcctcttccctctacaacactacttcccttcatccccctccccttaccaccacctcctgccgcctcctccccctcctccgctgccgccgcaccaccccgagctgaaccccaccaacatcgtcttcatcggccctcaagatccaccgtcgctgcacggatcgggaggtccatttctacaagaagaagaagaagaagaagaagaagaagaagaagaagcaaggatgtcccaaaatggtgggaagtgtttttcttcttgtgggttaattacaaaaaaaggcagtgtgaagaaagatcggcatagcaagatttacacagctcagggtttgagagacaggcgagtgagattgtccattgacatttctagaaagttctttgatcttcaagacatgttggggtttgacaaagctagcaaaaccctagattggctccttacaaagtcaaaaaaagccattaaagagttgacaaacaacaaaaatttgtataattttgactctgaatctgaggctaaccaaataatgttgcccaaatcctcaaacaaaacccttccatttcatgatgttttggccaaacaatctagggcaaaagcaagagcaagagctagggaaagaactaaggagaaaatcatgagcaagaatcgccaattccgatccccgccaccgccaccgccaccgccaccgcctccgccaccctcctcctcttcttcttccatacccgtcttcccaaaaggcggtaccgaccgttggcggcggagccaacttggatcaagaaaacaacatcatagggatcatgtggaagccttctcctatgatcacatcttcaaaaggagagtgttattacaacaattgcaacaatttctattttccacctccaaattgggacatcaatattgactcttttgccaaaccacaatctgggttttgttcaattgccaacatggattctctcccagctgcagcaacaatcaaacac

SEQ ID NO: 10-12 - skipped (3 entries)

SEQ ID NO: 13 - protein, 8 aa, synthetic construct
YNNCNNFY

SEQ ID NO: 14 - DNA, 21 bp, synthetic construct
  source 1..21: mol_type = other DNA
gcgttttaaaactttgagggg

SEQ ID NO: 15 - DNA, 22 bp, synthetic construct
  source 1..22: mol_type = other DNA
caaatgagatcatcaacatacc

SEQ ID NO: 16 - DNA, 23 bp, synthetic construct
  source 1..23: mol_type = other DNA
gtttcattaaaagtagtctaagg

SEQ ID NO: 17 - DNA, 19 bp, synthetic construct
  source 1..19: mol_type = other DNA
ttgaccttactggcacacg

SEQ ID NO: 18 - DNA, 19 bp, synthetic construct
  source 1..19: mol_type = other DNA
acaaacccaccgctactag

SEQ ID NO: 19 - DNA, 19 bp, synthetic construct
  source 1..19: mol_type = other DNA
gtatcagagccagtcactg

SEQ ID NO: 20 - DNA, 19 bp, synthetic construct
  source 1..19: mol_type = other DNA
ctagcagtctcacagggag

SEQ ID NO: 21 - DNA, 19 bp, synthetic construct
  source 1..19: mol_type = other DNA
tgttctcctccccaaccag

SEQ ID NO: 22 - DNA, 21 bp, synthetic construct
  source 1..21: mol_type = other DNA
ccattttggtccctatttaca

SEQ ID NO: 23 - DNA, 21 bp, synthetic construct
  source 1..21: mol_type = other DNA
ccattttggtctcttgctttc

SEQ ID NO: 24 - DNA, 22 bp, synthetic construct
  source 1..22: mol_type = other DNA
gacacacaaaatcaagaatcaa

SEQ ID NO: 25 - DNA, 18 bp, synthetic construct
  source 1..18: mol_type = other DNA
aaggcccatggagtgacc

SEQ ID NO: 26 - DNA, 22 bp, synthetic construct
  source 1..22: mol_type = other DNA
agatacacattttcagtttcac

SEQ ID NO: 27 - DNA, 20 bp, synthetic construct
  source 1..20: mol_type = other DNA
caaagctgccctaaaaacgg

SEQ ID NO: 28 - DNA, 23 bp, synthetic construct
  source 1..23: mol_type = other DNA
cataattggtgcttatattcaac

SEQ ID NO: 29 - DNA, 19 bp, synthetic construct
  source 1..19: mol_type = other DNA
cttcaactttgccatcccc

SEQ ID NO: 30 - DNA, 22 bp, synthetic construct
  source 1..22: mol_type = other DNA
ttctgaagatttatgttttgcc

SEQ ID NO: 31 - DNA, 21 bp, synthetic construct
  source 1..21: mol_type = other DNA
tctgttctaaaagccattctc

SEQ ID NO: 32 - DNA, 21 bp, synthetic construct
  source 1..21: mol_type = other DNA
cacattacaaacctaaagacc

SEQ ID NO: 33 - DNA, 20 bp, synthetic construct
  source 1..20: mol_type = other DNA
tctttccctagctcttgctc

SEQ ID NO: 34 - DNA, 676 bp, synthetic construct
  source 1..676: mol_type = other DNA
cacattacaaacctaaagaccctaaacttaccccaaaaccacacaaaaaaacatgttttcttccaccaacaatctccacctcttccctcttcaacactacttcccttcatccccctccccttaccaccacctcctgccgcctcctccccctcctccgctgccgccgcaccaccccgagctgaaccccaccaacatcgtcttcatcggccctcaagatccaccgtcgctgcacggatcgggaggtccatttctacaagaagaagaagaagaagaagaagaagaagcaaggatgtcccaaaatggtgggaagtgtttttctccttgtgggttaattacaaaaaaaggcagtgtgaagaaagatcgacatagcaagatttacacagctcagggtttgagagacaggcgagtgagattgtccattgacatttctagaaagttctttgatcttcaagacatgttggggtttgacaaagctagcaaaaccctagattggctccttacaaagtcaaaaaaagccattaaagagttgacaaacaacaaaaatttgtataattttgactctgaatctgaggctaaccaaataatgttgcccaaatcctcaaacaaaacccttccatttcatgatgttttggccaaacaatctagggcaaaagctagagcaagagctagggaaaga

SEQ ID NO: 35 - DNA, 21 bp, synthetic construct
  source 1..21: mol_type = other DNA
cttcacatctgcccattgttc

SEQ ID NO: 36 - DNA, 20 bp, synthetic construct
  source 1..20: mol_type = other DNA
aagaccaaggcaagaacacc

SEQ ID NO: 37 - DNA, 19 bp, synthetic construct
  source 1..19: mol_type = other DNA
tcgtcaggagggaagaaac

SEQ ID NO: 38 - DNA, 19 bp, synthetic construct
  source 1..19: mol_type = other DNA
aggggcttctcagcgattg

SEQ ID NO: 39 - DNA, 20 bp, synthetic construct
  source 1..20: mol_type = other DNA
acaagccgtaaggatagagg

SEQ ID NO: 40 - DNA, 22 bp, synthetic construct
  source 1..22: mol_type = other DNA
agaaccaacaaaatagatcacc

SEQ ID NO: 41 - DNA, 20 bp, synthetic construct
  source 1..20: mol_type = other DNA
cacattcctccataaagttc

SEQ ID NO: 42 - DNA, 21 bp, synthetic construct
  source 1..21: mol_type = other DNA
atttcaatttctctcttctac

SEQ ID NO: 43 - DNA, 24 bp, synthetic construct
  source 1..24: mol_type = other DNA
ttaattgctatttgaagtattgag

SEQ ID NO: 44 - DNA, 22 bp, synthetic construct
  source 1..22: mol_type = other DNA
tcgttctaaattacttgctatg

SEQ ID NO: 45 - DNA, 23 bp, synthetic construct
  source 1..23: mol_type = other DNA
attttctcatttattttaccaac

SEQ ID NO: 46 - DNA, 20 bp, synthetic construct
  source 1..20: mol_type = other DNA
gccattgccccaaatttctt

SEQ ID NO: 47 - DNA, 20 bp, synthetic construct
  source 1..20: mol_type = other DNA
ctagcaagaggagatgtcac

SEQ ID NO: 48 - DNA, 22 bp, synthetic construct
  source 1..22: mol_type = other DNA
agtttgatttttgggacttacc

SEQ ID NO: 49 - DNA, 22 bp, synthetic construct
  source 1..22: mol_type = other DNA
cctttgtgacacattatttaag

SEQ ID NO: 50 - DNA, 20 bp, synthetic construct
  source 1..20: mol_type = other DNA
catgtcttgttcgttgttcc

SEQ ID NO: 51 - DNA, 23 bp, synthetic construct
  source 1..23: mol_type = other DNA
gtatgtatgtaattagcattgtg

SEQ ID NO: 52 - DNA, 21 bp, synthetic construct
  source 1..21: mol_type = other DNA
aatctgatgatggcaacttgc

SEQ ID NO: 53 - DNA, 20 bp, synthetic construct
  source 1..20: mol_type = other DNA
cttgctcgttatgctgattc

SEQ ID NO: 54 - DNA, 20 bp, synthetic construct
  source 1..20: mol_type = other DNA
atgctcaatccaccataagg

SEQ ID NO: 55 - DNA, 20 bp, synthetic construct
  source 1..20: mol_type = other DNA
tccatacagtagtgctaagg

SEQ ID NO: 56 - DNA, 22 bp, synthetic construct
  source 1..22: mol_type = other DNA
ctgctttatgattgaatctatg

SEQ ID NO: 57 - DNA, 20 bp, synthetic construct
  source 1..20: mol_type = other DNA
tcaagcctgcaagatgtagc

SEQ ID NO: 58 - DNA, 21 bp, synthetic construct
  source 1..21: mol_type = other DNA
gcattccacacccttataaag

SEQ ID NO: 59 - DNA, 23 bp, synthetic construct
  source 1..23: mol_type = other DNA
tcacaatagaagtcatttaatac

SEQ ID NO: 60 - DNA, 21 bp, synthetic construct
  source 1..21: mol_type = other DNA
ccattttggtctcttgctttc

SEQ ID NO: 61 - DNA, 20 bp, synthetic construct
  source 1..20: mol_type = other DNA
gcactgtgtatggacttctc

SEQ ID NO: 62 - DNA, 23 bp, synthetic construct
  source 1..23: mol_type = other DNA
gattctcaatgattaaaaacatg

SEQ ID NO: 63 - DNA, 21 bp, synthetic construct
  source 1..21: mol_type = other DNA
tatgtgcgcagacctattttc

SEQ ID NO: 64 - DNA, 21 bp, synthetic construct
  source 1..21: mol_type = other DNA
atgttcttcgtagttctgttc

SEQ ID NO: 65 - DNA, 20 bp, synthetic construct
  source 1..20: mol_type = other DNA
gttcccagccatcattttcc

SEQ ID NO: 66 - DNA, 20 bp, synthetic construct
  source 1..20: mol_type = other DNA
tcagcacattcacgtccaac

SEQ ID NO: 67 - DNA, 20 bp, synthetic construct
  source 1..20: mol_type = other DNA
catggaagcttactacacag

SEQ ID NO: 68 - DNA, 22 bp, synthetic construct
  source 1..22: mol_type = other DNA
ctgttgttagattcttaagatg

SEQ ID NO: 69 - DNA, 21 bp, synthetic construct
  source 1..21: mol_type = other DNA
gtgttattaccaggatggatc

SEQ ID NO: 70 - DNA, 20 bp, synthetic construct
  source 1..20: mol_type = other DNA
ctgaactgtacttcgagtcg

SEQ ID NO: 71 - DNA, 24 bp, synthetic construct
  source 1..24: mol_type = other DNA
tatgattaatggaaagttgatttg

SEQ ID NO: 72 - DNA, 21 bp, synthetic construct
  source 1..21: mol_type = other DNA
gttcgaggttaatcttgaatc

SEQ ID NO: 73 - DNA, 23 bp, synthetic construct
  source 1..23: mol_type = other DNA
ggcatcacaaaattaaaatgtag

SEQ ID NO: 74 - DNA, 18 bp, synthetic construct
  source 1..18: mol_type = other DNA
catagccccaacccaacc